(19) United States
(12) Patent Application Publication
(10) Pub. No.: US 2025/0104808 A1
(43) Pub. Date: Mar. 27, 2025

(54) DETERMINING B-ALLELE FREQUENCY VALUES FROM GENOME MAPPING DATA

(71) Applicant: BIONANO GENOMICS, INC., San Diego, CA (US)

(72) Inventor: Soheil SHAMS, San Diego, CA (US)

(21) Appl. No.: 18/973,053

(22) Filed: Dec. 8, 2024

Related U.S. Application Data
(63) Continuation-in-part of application No. PCT/US2023/068138, filed on Jun. 8, 2023.
(60) Provisional application No. 63/350,378, filed on Jun. 8, 2022.
(60) Provisional application No. 63/414,860, filed on Oct. 10, 2022.

(51) Int. Cl. G16B 20/20

(52) U.S. Cl. CPC G16B 20/20

(57) ABSTRACT

Disclosed herein includes systems, devices, and methods for determining a B-allele frequency (BAF) value (e.g., a normalized BAF value) of a SNP for a test sample obtained from a test subject (e.g., a human subject) from genome mapping (GM) data, such as optical genome mapping (OGM) data or electronic genome mapping (EGM) data.

### BACKGROUND

### Field

This disclosure relates generally to the field of genome mapping (e.g., optical genome mapping), and more particularly to determining a B-allele frequency (BAF) value (e.g., a normalized BAF value) from genome mapping data (e.g., OGM data).

### Background

Genome mapping, such as Optical Genome Mapping (OGM), is a technology which can evaluate the labeling pattern (such as fluorescent labeling pattern for OGM) of individual DNA molecules to perform an unbiased assessment of genome-wide structural variants. The specific labeling profile of individual DNA molecules, including spacing and pattern of labels (labels for OGM can be hexamers labels), can be grouped based on similarity to produce consensus maps which can be compared in silico to the expected labeling pattern of a reference genome. There is a need to determine a B-allele frequency (BAF) value (e.g., a normalized BAF value) from genome mapping data (e.g., OGM data).

### SUMMARY

Disclosed herein include methods for determining a B-allele frequency (BAF) value (e.g., a normalized BAF value) from genome mapping (GM) data. In some embodiments, a method for determining a normalized BAF value from GM data is under control of a processor (e.g., a hardware processor or a virtual processor) and comprises: receiving genome mapping (GM) data generated from a plurality of control samples obtained from a plurality of control subjects. The method can comprise: determining a B-allele frequency (BAF) value of a single nucleotide polymorphism (SNP) of a gene (or a SNP in or relative to a reference genome sequence) for each of the plurality of control samples using the GM data generated from control sample. The method can comprise: clustering the BAF values of the SNP of the gene for control samples of the plurality of control samples into a plurality of clusters each comprising (or has or is associated with) a cluster center. The method can comprise: receiving GM data generated from a test sample obtained from a test subject. The method can comprise: determining a BAF value of the SNP of the gene for the test sample using the GM data of the test sample. The method can comprise: determining a normalized BAF value of the SNP of the gene for the test sample from the BAF value of the SNP of the gene for the test sample using one or more (e.g., 2, 3, 4, 5, or more) of the cluster centers (or using values of one or more of the cluster centers). In some embodiments, the GM data comprises optical genome mapping (OGM) data. In some embodiments, the GM data comprises electronic genome mapping (EGM) data.

Disclosed herein include methods for determining B-allele frequency (BAF) values (e.g., a normalized BAF values) from genome mapping (GM) data. In some embodiments, a method for determining a normalized BAF value from GM data is under control of a processor (or a hardware processor or a virtual processor) and comprises: receiving genome mapping (GM) data generated from a plurality of control samples obtained from a plurality of control subjects. The method can comprise: determining a B-allele frequency (BAF) value of each single nucleotide polymorphism (SNP) of SNPs of a plurality of SNPs (e.g., SNPs in or relative to a reference genome sequence) for each of the plurality of control samples using the GM data generated from control sample. The method can comprise: clustering the BAF values of the SNP for control samples of the plurality of control samples into a plurality of clusters each comprising a cluster center. The method can comprise: receiving GM data generated from a test sample obtained from a test subject. The method can comprise: determining a BAF value of the SNP for the test sample using the GM data of the test sample. The method can comprise: determining a normalized BAF value of the SNP for the test sample from the BAF value of the SNP for the test sample using one or more (e.g., 2, 3, 4, 5, or more) of the cluster centers (or values of one or more of the cluster centers). In some embodiments, the GM data comprises optical genome mapping (OGM) data. In some embodiments, the GM data comprises electronic genome mapping (EGM) data.

In some embodiments, each control sample can be obtained from a different control subject. Two control samples can be obtained from one control subject. The number of the control samples can be, for example, 20, 30, 40, 50, 60, 70, 80, 90, 100, 200, 300, 400, 500, 750, 1000, 1500, 2500, 5000, 7500, 10000, or more. The number of the control subjects can be, for example, 20, 30, 40, 50, 60, 70, 80, 90, 100, 200, 300, 400, 500, 750, 1000, 1500, 2500, 5000, 7500, 10000, or more. In some embodiments, receiving the GM data generated from the plurality of control samples obtained from the plurality of control subjects comprises: generating the GM data from a control sample obtained from a control subject. In some embodiments, the GM data generated from a control sample obtained from a control subject comprises a deoxyribonucleic acid (DNA) consensus map for the control subject. The DNA consensus map can comprise presence and/or absence of labels (or signals) at (or corresponding to, from, or mapped to) the position of the SNP. For EGM, signals can be electric signals. The presence of a label (which can be a non-fluorescent label) can result in a change in the electric signal. For OGM, labels can be fluorescent labels, and signals can be fluorescent signals. For example, the DNA consensus map can comprise presence and/or absence of fluorescent labels (or fluorescent signals) at (or corresponding to, from, or mapped to) the position of the SNP.

In some embodiments, a label for GM is attached to a predetermined sequence. For example, a fluorescent label for OGM can be attached to a predetermined sequence. The gene (or a reference genome sequence) can comprise the predetermined sequence. The SNP can be present at a position (e.g., position 1, 2, 3, 4, 5, 6, 7, 8, 9, or 10) in the predetermined sequence in the gene (or a reference genome sequence). The SNP can overlap the predetermined sequence in the gene (or a reference genome sequence). The nucleobase at the position in the predetermined sequence corresponds to (or is) an A-allele of the SNP. The presence of the label (or signal) at the SNP in the GM data indicates an A-allele of the SNP. For example, the presence of the fluorescent label at the SNP in the OGM data indicates an A-allele of the SNP. The absence of the label (or signal) at the SNP in the GM data indicates a B-allele of the SNP. For example, the absence of the fluorescent label at the SNP in the OGM data indicates a B-allele of the SNP. The predetermine sequence can six nucleotides (or 5, 6, 7, 8, 9, 10, or more nucleotides) in length. The predetermine sequence can comprise 5′-CTTAAG-3′. The predetermined sequence can be a recognition sequence of a methyltransferase. The methyltransferase can be a direct labeling enzyme (DLE-1).

In some embodiments, the method can comprise: determining the plurality of SNPs. Each of the plurality of SNPs can overlap the predetermined sequence. The plurality of SNPs can comprise one, some, or all SNPs present in a reference genome sequence that overlap the predetermined sequence. The species of the test subject and a species of a control subject (or each control subject) can be identical. The reference genome sequence can be that of a species (e.g., a vertebrate, a mammal, or a human) of the test subject (or a control subject), such as a reference human genome sequence (e.g., hg38 (GRCh38), hg19 (GRCh37), hg18, hg17, hg16). The plurality of SNPs can comprise some or all SNPs in a reference genome sequence with a minor allele frequency (MAF) of more than a predetermined percentage threshold, such as 15% (or 5%, 6%, 7%, 8%, 9%, 10%, 11%, 12%, 13%, 14%, 15%, 16%, 17%, 18%, 19%, 20%, 21%, 22%, 23%, 24%, or 25%). The plurality of SNPs can comprise or comprise about 5000, 6000, 7000, 8000, 9000, 10000, 11000 (e.g., 11724), 12000, 13000, 14000, 15000, 16000, 17000, 18000, 19000, 20000, 22500, 25000, 27500, 30000, 40000, 50000, or more, SNPs.

In some embodiments, a BAF value is or is about 0, 0.05, 0.1, 0.2, 0.3, 0.4, 0.5, 0.6, 0.7, 0.8, 0.9, 0.95, or 1 (on a scale of 0 to 1 where AA, AB, and BB genotypes have BAF values of 0, 0.5, and 1 respectively in an idealized situation). In some embodiments, determining the BAF value of the SNP for each of the plurality of control samples comprises: determining the BAF value of the SNP using absence (and/or presence) of a label (or signal) at (or corresponding to, from, or mapped to) the position of the SNP in the GM data generated from the control sample. For EGM, signals can be electric signals. The presence of a label (which can be a non-fluorescent label) can result in a change in the electric signal. For OGM, labels can be fluorescent labels, and signals can be fluorescent signals. For example, determining the BAF value of the SNP for each of the plurality of control samples comprises: determining the BAF value of the SNP using absence (and/or presence) of a fluorescent label (or fluorescent signal) at (or corresponding to, from, or mapped to) the position of the SNP in the OGM data generated from the control sample. In some embodiments, determining the BAF value of the SNP for each of the plurality of control samples comprises: determining a signal strength of a B-allele of the SNP in the GM data generated from the control sample (or the GM data for the control sample). For example, determining the BAF value of the SNP for each of the plurality of control samples comprises: determining a signal strength of a B-allele of the SNP in the OGM data generated from the control sample (or the OGM data for the control sample). Determining the BAF value of the SNP for each of the plurality of control samples can comprise: determining the BAF value of the SNP for the control sample using the signal strength of the B-allele of the SNP for the control sample. Determining the signal strength of the B-allele of the SNP in the GM data generated from the control sample can comprise: determining the signal strength of the B-allele of the SNP in the GM data generated from the control sample using absence (and/or presence) of a label (or signal) at the position of the SNP in the GM data generated from the control sample. For example, determining the signal strength of the B-allele of the SNP in the OGM data generated from the control sample can comprise: determining the signal strength of the B-allele of the SNP in the OGM data generated from the control sample using absence (and/or presence) of a fluorescent label (or fluorescent signal) at the position of the SNP in the OGM data generated from the control sample. The signal strength of the B-allele of the SNP for the control sample can be a ratio of (i) a number of deoxyribonucleic acid (DNA) molecules (or fragments) comprising the SNP and without a label at the position of the SNP in the GM data generated from the control sample and (ii) a number of DNA molecules (or fragments) comprising the SNP in the GM data generated from the control sample. For example, the signal strength of the B-allele of the SNP for the control sample can be a ratio of (i) a number of deoxyribonucleic acid (DNA) molecules (or fragments) comprising the SNP and without a fluorescent label at the position of the SNP in the OGM data generated from the control sample and (ii) a number of DNA molecules (or fragments) comprising the SNP in the OGM data generated from the control sample. The signal strength of the B-allele of the SNP for the control sample can be a percentage of deoxyribonucleic acid (DNA) molecules (or fragments) comprising the SNP in the GM data generated from the control sample and without a label at the position of the SNP. For example, the signal strength of the B-allele of the SNP for the control sample can be a percentage of deoxyribonucleic acid (DNA) molecules (or fragments) comprising the SNP in the OGM data generated from the control sample and without a fluorescent label at the position of the SNP. The signal strength of the B-allele of the SNP for the control sample can be 1 minus a ratio of (i) a number of DNA molecules (or fragments) comprising the SNP and with a label at the position of the SNP in the GM data generated from the test sample and (ii) a number of DNA molecules (or fragments) comprising the SNP in the GM data generated from the test sample. For example, the signal strength of the B-allele of the SNP for the control sample can be 1 minus a ratio of (i) a number of DNA molecules (or fragments) comprising the SNP and with a fluorescent label at the position of the SNP in the OGM data generated from the test sample and (ii) a number of DNA molecules (or fragments) comprising the SNP in the OGM data generated from the test sample. The signal strength of the B-allele of the SNP for the control sample can be 1 minus a percentage of DNA molecules (or fragments) comprising the SNP in the GM data generated from the control sample and with a label at the position of the SNP. For example, the signal strength of the B-allele of the SNP for the control sample can be 1 minus a percentage of DNA molecules (or fragments) comprising the SNP in the OGM data generated from the control sample and with a fluorescent label at the position of the SNP. A (or each) DNA molecule (or fragment) can be about, or at least, 150 kilobases (kbp) in length (such as 250 kbp, 500 kbp, 750 kbp, 1 megabases (Mbp), 2 Mbp, or longer, in length). A (or each) DNA molecule (or fragment) can comprise of at least 7, 8, 9, 10, 11, 12, 13, 14, 15, 16, 17, 18, 19, 20, 25, 30, 40, 50, 75, 100, or more) labels. Labels can be fluorescent labels for OGM. Labels can be non-fluorescent labels for EGM.

In some embodiments, the method comprises: determining a separation between a pair (or each pair) of clusters of the plurality of clusters for a second SNP (e.g., a low-quality SNP) of the SNPs (or the plurality of SNPs) is below a separation threshold. The separation threshold can be, for example, 0.15, 0.2, 0.25, 0.3, 0.35, 0.4, or 0.45 on a scale where AA, AB, and BB genotypes have BAF values of 0, 0.5, and 1 respectively in an idealized situation. The separation can comprise a Silhouette score, which can be between −1 and 1. The separation threshold can be 0.2, 0.3, 0.4, 0.5, 0.6, 0.7, 0.8, or 0.9. The method can comprise: removing the second SNP from BAF value and normalized BAF value determination. The method can comprise: calculating a loss of heterozygosity (LOH) without using the second SNP, without using the BAF value of the second SNP, and/or without using the normalized BAF value of the second SNP. In some embodiments, the method comprises: calculating a loss of heterozygosity (LOH) for the test sample using the normalized BAF values of two or more of the SNPs (or the plurality of SNPs).

In some embodiments, the method can comprise: performing split-label enhancement. For example, a label can be assigned to two reference label positions for at least a predetermined percentage (e.g., 5%, 6%, 7%, 8%, 9%, 10%, 11%, 12%, 13%, 14%, 15%, or more) of DNA molecules comprising a third SNP (or split label SNP) the SNPs (or the plurality of SNPs). Such assignment can be present in the GM data generated from a control sample. Such assignment can be present in the GM data generated from two or more of the plurality of control samples. Such assignment can be present in the GM data generated from each of the plurality of control samples. Such assignment can be present in the GM data generated from the test sample. The method can comprise: removing the third SNP from BAF value and normalized BAF value determination. The method can comprise: calculating a loss of heterozygosity (LOH) without using the third SNP, the BAF value of the third SNP, and/or the normalized BAF value of the third SNP.

In some embodiments, the SNP is at a region with a copy number (CN) loss or a copy number gain. The SNP can be at a region with 0% loss of one copy. The SNP can be at a region with 50% loss of one copy. The SNP can be at a region with complete loss of one copy. The SNP can be at a region with 50% trisomy. The SNP can be at a region with complete trisomy. The SNP can be at a region with complete tetrasomy. Determining the BAF value of the SNP for each of the plurality of control samples can comprise: determining a signal strength of a B-allele of the SNP in the GM data generated from the control sample (or the GM data for the control sample) using a number of deoxyribonucleic acid (DNA) molecules comprising the SNP mapped to a loss map, a number of DNA molecules comprising the SNP mapped to all maps, and/or a number of DNA molecules mapped to a gain/duplicate map. Determining the BAF value of the SNP for each of the plurality of control samples can comprise: determining the BAF value of the SNP for the control sample using the signal strength of the B-allele of the SNP for the control sample.

In some embodiments, the plurality of clusters can comprise 2, 3, 4, 5, 6, 7, 8, 9, 10 or more clusters. A cluster can comprise 0, 1, 2, 3, 4, 5, 6, 7, 8, 9, 10, 20, 30, 40, 50, 75, 100, 250, 500, 750, 1000, 2500, 5000, 10000, 25000, 50000, or more BAF values. A cluster center can have a value of, or of about, 0, 0.05, 0.1, 0.2, 0.3, 0.4, 0.5, 0.6, 0.7, 0.8, 0.9, 0.95, or 1 (on a scale of 0 to 1 where AA, AB, and BB genotypes have BAF values of 0, 0.5, and 1 respectively in an idealized situation).

In some embodiments, clustering the BAF values comprises: clustering the BAF values of the SNP for control samples of the plurality of control samples into the plurality of clusters using connectivity-based clustering (e.g., hierarchical clustering), centroid-based clustering (e.g., k-means clustering), distribution-based clustering (e.g., Gaussian mixture model clustering), density-based clustering, grid-based clustering, or a combination thereof. The clustering can be based on a connectivity model (e.g., hierarchical clustering), a centroid model (e.g., k-means clustering), a distribution model (e.g., expectation-maximization), a density model (e.g., DBSCAN and OPTICS), a subspace model (e.g., biclustering), a group model, a graph-based model, a signed graph model, a neural model (e.g., unsupervised neural network), Principal Component Analysis, Independent Component Analysis, or a combination thereof.

In some embodiments, the plurality of clusters comprises three clusters representing AA, AB, and BB genotypes of the SNP. The three cluster centers of the three clusters representing AA, AB, and BB genotypes can be at about 0, 0.5, and 1.0 respectively. The three cluster centers representing AA, AB, and BB may not be at 0, 0.5, and 1.0 respectively. In some embodiments, the method comprises: determining a cluster center of each of the plurality of clusters. In some embodiments, a cluster center of a cluster of the plurality of cluster is an average, a mean, a median, or a combination thereof, of the BAF values in the cluster. In some embodiments, a cluster of the plurality of clusters representing BB genotype for a SNP comprises an insufficient number of BAF values (e.g., 20, 19, 18, 17, 16, 15, 14, 13, 12, 11, 10, 9, 8, 7, 6, 5, 4, 3, 2 1, or 0 BAF values). The cluster center of the cluster comprising an insufficient number of BAF values can comprise a measure of cluster centers representing BB genotypes for two or more (e.g., 3, 4, 5, 6, 7, 8, 9, 10, or more, or all) of the SNPs (or the plurality of SNPs) with sufficient numbers of BAF values. The measure of cluster centers representing BB genotypes can be an average, a mean, a median, or a combination thereof, of the cluster centers representing BB genotypes.

In some embodiments, receiving the GM data generated from the test sample obtained from the test subject comprises: generating the GM data from the test sample obtained from the test subject. In some embodiments, the GM data generated from the test sample obtained from the test subject comprises a deoxyribonucleic acid (DNA) consensus map for the test subject. The DNA consensus map can comprise presence and/or absence of labels (or signals) at (or corresponding to, from, or mapped to) the position of the SNP. For OGM, labels can be fluorescent labels, and signals can be fluorescent signals. For EGM, signals can be electric signals. The presence of a label (which can be a non-fluorescent label) can result in a change in the electric signal.

In some embodiments, a BAF value is or is about 0, 0.05, 0.1, 0.2, 0.3, 0.4, 0.5, 0.6, 0.7, 0.8, 0.9, 0.95, or 1 (on a scale of 0 to 1 where AA, AB, and BB genotypes have BAF values of 0, 0.5, and 1 respectively in an idealized situation). In some embodiments, determining the BAF value of the SNP for the test sample comprises: determining the BAF value of the SNP using absence (and/or presence) of a label (or signal) at (or corresponding to, from, or mapped to) the position of the SNP in the GM data generated from the test sample. For EGM, signals can be electric signals. The presence of a label (which can be a non-fluorescent label) can result in a change in the electric signal. For OGM, labels can be fluorescent labels, and signals can be fluorescent signals. In some embodiments, determining the BAF value of the SNP for the test sample comprises: determining a signal strength of a B-allele of the SNP in the GM data generated from the test sample (or in the GM data for the test sample). For example, determining the BAF value of the SNP for the test sample comprises: determining a signal strength of a B-allele of the SNP in the OGM data generated from the test sample (or in the OGM data for the test sample). Determining the BAF value of the SNP for the test sample can comprise: determining the BAF value of the SNP for the test sample using the signal strength of the B-allele of the SNP for the test sample. In some embodiments, determining the signal strength of the B-allele of the SNP in the GM data generated from the test sample comprises: determining the signal strength of the B-allele of the SNP in the GM data generated from the test sample using absence (and/or presence) of a label (or signal) at the position of the SNP in the GM data generated from the test sample. For example, determining the signal strength of the B-allele of the SNP in the OGM data generated from the test sample comprises: determining the signal strength of the B-allele of the SNP in the OGM data generated from the test sample using absence (and/or presence) of a fluorescent label (or fluorescent signal) at the position of the SNP in the OGM data generated from the test sample. The signal strength of the B-allele of the SNP for the test sample can be a ratio of (i) a number of deoxyribonucleic acid (DNA) molecules comprising the SNP and without a label at the position of the SNP in the GM data generated from the test sample and (ii) a number of DNA molecules comprising the SNP in the GM data generated from the test sample. For example, the signal strength of the B-allele of the SNP for the test sample can be a ratio of (i) a number of deoxyribonucleic acid (DNA) molecules comprising the SNP and without a fluorescent label at the position of the SNP in the OGM data generated from the test sample and (ii) a number of DNA molecules comprising the SNP in the OGM data generated from the test sample. The signal strength of the B-allele of the SNP for the test sample can be a percentage of DNA molecules comprising the SNP in the GM data generated from the test sample and without a label at the position of the SNP. For example, the signal strength of the B-allele of the SNP for the test sample can be a percentage of DNA molecules comprising the SNP in the OGM data generated from the test sample and without a fluorescent label at the position of the SNP. The signal strength of the B-allele of the SNP for the test sample can be 1 minus a ratio of (i) a number of DNA molecules comprising the SNP and with a label at the position of the SNP in the GM data generated from the test sample and (ii) a number of DNA molecules comprising the SNP in the GM data generated from the test sample. For example, the signal strength of the B-allele of the SNP for the test sample can be 1 minus a ratio of (i) a number of DNA molecules comprising the SNP and with a fluorescent label at the position of the SNP in the OGM data generated from the test sample and (ii) a number of DNA molecules comprising the SNP in the OGM data generated from the test sample. The signal strength of the B-allele of the SNP for the test sample can be 1 minus a percentage of DNA molecules comprising the SNP in the GM data generated from the test sample and with a label at the position of the SNP. For example, the signal strength of the B-allele of the SNP for the test sample can be 1 minus a percentage of DNA molecules comprising the SNP in the OGM data generated from the test sample and with a fluorescent label at the position of the SNP.

In some embodiments, determining the BAF value of the SNP for the test sample comprises: determining a signal strength of a B-allele of the SNP in the GM data generated from the test sample (or GM data for the test sample) using a number of deoxyribonucleic acid (DNA) molecules comprising the SNP mapped to a loss map, a number of DNA molecules comprising the SNP mapped to all maps, and/or a number of DNA molecules mapped to a gain/duplicate map. Determining the BAF value of the SNP for the test sample can comprise: determining the BAF value of the SNP for the test sample using the signal strength of the B-allele of the SNP for the test sample.

In some embodiments, a normalized BAF value can be, or be about, 0, 0.05, 0.1, 0.2, 0.3, 0.4, 0.5, 0.6, 0.7, 0.8, 0.9, 0.95, or 1 (on a scale of 0 to 1 where AA, AB, and BB genotypes have BAF values of 0, 0.5, and 1 respectively in an idealized situation). In some embodiments, determining the normalized BAF value for the test sample comprises: determining the normalized BAF value of the SNP for the test sample from the BAF value of the SNP for the test sample using two of the cluster centers (or values of two of the cluster centers). In some embodiments, the BAF value of the SNP is smaller than the cluster center of the cluster representing AB genotype. The normalized BAF value of the SNP can be a ratio of (i) a distance between the cluster center of the cluster representing AA genotype and the BAF value of the SNP, and (ii) two times a distance between the cluster center of the cluster representing AA genotype and between the cluster center of the cluster representing AB genotype. In some embodiments, the BAF value of the SNP is greater than the cluster center of the cluster representing AB genotype. The normalized BAF value of the SNP can be one minus a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

In some embodiments, the method can comprise performing per sample adjustment. For example, corresponding clusters of the pluralities of clusters can represent a genotype. The method can further comprise: clustering BAF values of SNPs for the test sample into a plurality of test sample clusters, representing the genotypes, each comprising a test sample cluster center. The method can further comprise: determining a measure of the cluster centers of the clusters representing each of the genotypes. The measure of the cluster centers can be an average, a mean, a median, or a combination thereof, of the cluster centers. The method can further comprise: determining a difference between a (or each) test sample cluster center and the measure of the cluster centers of the clusters representing an identical genotype. The method can further comprise: for a (or each) SNP of the SNPs, adjusting a (or each) cluster center of the cluster of the plurality of clusters for the SNP based on the difference to determine an adjusted cluster center. Determining the normalized BAF value of the SNP for the test sample can comprise: determining the normalized BAF value of the SNP for the test sample from the BAF value of the SNP for the test sample using one or more of the plurality of adjusted cluster centers. In some embodiments, the plurality of test sample clusters comprises three test sample clusters representing AA, AB, and BB genotypes of the SNP.

In some embodiments, the method comprises: creating a file or a report and/or generating a user interface (UI) comprising a UI element. The UI element can represent or comprise (i) the BAF value of the SNP for one, one or more, or each, of the plurality of control samples, (ii) the signal strength of the B-allele of the SNP for one, one or more, or each, of the plurality of control samples, (iii) the BAF value of the SNP for the test sample, (iv) the signal strength of the B-allele of the SNP for the test sample, and/or (v) the normalized BAF value of the SNP for the test sample. The UI element can comprise a plot representing some or all of the values and signal strengths. In some embodiments, a control sample, or a test sample, comprises cells, cell-free DNA, cell-free fetal DNA, amniotic fluid, a blood sample, a bone marrow sample, a biopsy sample, or a combination thereof.

Disclosed herein include systems for determining a B-allele frequency (BAF) value, or a normalized BAF value, from genome mapping (GM) data. In some embodiments, a system for determining a normalized BAF value from GM data comprises non-transitory memory configured to store: executable instructions. The non-transitory memory can be configured to store: genome mapping (GM) data generated from a plurality of control samples obtained from a plurality of control subjects. The non-transitory memory can be configured to store: a B-allele frequency (BAF) value of a single nucleotide polymorphism (SNP) of a gene (or in a reference genome sequence) for each of the plurality of control samples determined using the GM data generated from control sample. The non-transitory memory can be configured to store: a plurality of clusters, each comprising a cluster center, generated from BAF values of the SNP of the gene for control samples of the plurality of control samples. The system can comprise: a processor (e.g., a hardware processor or a virtual processor) in communication with the non-transitory memory. The processor can be programmed by the executable instructions to perform: receiving GM data generated from a test sample obtained from a test subject. The processor can be programmed by the executable instructions to perform: determining a BAF value of the SNP of the gene for the test sample using the GM data of the test sample. The processor can be programmed by the executable instructions to perform: determining a normalized BAF value of the SNP of the gene for the test sample from the BAF value of the SNP of the gene for the test sample using one or more of the cluster centers (or values of one or more of the cluster centers). In some embodiments, the processor is programmed by the executable instructions to perform: receiving the GM data generated from the plurality of control samples obtained from the plurality of control subjects. The processor can be programmed by the executable instructions to perform: determining the BAF value of the SNP of the gene (or a reference genome sequence) for each of the plurality of control samples using the GM data generated from control sample. The processor can be programmed by the executable instructions to perform: clustering BAF values of the SNP of the gene for control samples of the plurality of control samples into a plurality of clusters, each comprising a cluster center. In some embodiments, the GM data comprises optical genome mapping (OGM) data. In some embodiments, the GM data comprises electronic genome mapping (EGM) data.

Disclosed herein include systems for determining B-allele frequency (BAF) values (e.g., a normalized BAF values) from genome mapping (GM) data. In some embodiments, a system for determining a BAF value from GM data comprises: non-transitory memory configured to store: executable instructions. The non-transitory memory can be configured to store: genome mapping (GM) data generated from a plurality of control samples obtained from a plurality of control subjects. The non-transitory memory can be configured to store: a B-allele frequency (BAF) value of each single nucleotide polymorphism (SNP) of SNPs of a plurality of SNPs (e.g., a plurality of SNPs in a reference genome sequence) for each of the plurality of control samples determined using the GM data generated from control sample. The non-transitory memory can be configured to store: a plurality of clusters, each comprising a cluster center, generated from BAF values of the SNP for control samples of the plurality of control samples. The system can comprise: a processor (e.g., a hardware processor or a virtual processor) in communication with the non-transitory memory. The processor can be programmed by the executable instructions to perform: clustering BAF values of the SNP for control samples of the plurality of control samples into a plurality of clusters each comprising a cluster center. The processor can be programmed by the executable instructions to perform: receiving GM data generated from a test sample obtained from a test subject. The processor can be programmed by the executable instructions to perform: determining a BAF value of the SNP for the test sample using the GM data of the test sample. The processor can be programmed by the executable instructions to perform: determining a normalized BAF value of the SNP for the test sample from the BAF value of the SNP for the test sample using one or more of the plurality of cluster centers. In some embodiments, wherein the processor is programmed by the executable instructions to perform: receiving the GM data generated from the plurality of control samples obtained from the plurality of control subjects. The processor can be programmed by the executable instructions to perform: determining the BAF value of each SNP of the SNPs of the plurality of SNPs for each of the plurality of control samples using the GM data generated from control sample. The processor can be programmed by the executable instructions to perform: clustering BAF values of the SNP for control samples of the plurality of control samples into a plurality of clusters each comprising a cluster center. In some embodiments, the GM data comprises optical genome mapping (OGM) data. In some embodiments, the GM data comprises electronic genome mapping (EGM) data.

In some embodiments, receiving the GM data generated from the plurality of control samples obtained from the plurality of control subjects comprises: generating the GM data from a control sample obtained from a control subject. In some embodiments, the GM data generated from a control sample obtained from a control subject comprises a deoxyribonucleic acid (DNA) consensus map for the control subject, optionally wherein the DNA consensus map comprises presence and/or absence of labels at the position of the SNP. For OGM, labels can be fluorescent labels, and signals can be fluorescent signals. For EGM, signals can be electric signals. The presence of a label (which can be a non-fluorescent label) can result in a change in the electric signal.

In some embodiments, a label for GM is attached to a predetermined sequence. The gene can comprise the predetermined sequence. The SNP can be present at a position in the predetermined sequence in the gene. The nucleobase at the position in the predetermined sequence can correspond to an A-allele of the SNP. The predetermine sequence can be six nucleotides in length. The predetermine sequence can comprise 5′-CTTAAG-3′. The predetermined sequence can be a recognition sequence of a methyltransferase. The methyltransferase can comprise a direct labeling enzyme (DLE-1).

In some embodiments, the processor is programmed by the executable instructions to perform: determining the plurality of SNPs. Each of the plurality of SNPs can overlap the predetermined sequence. In some embodiments, the plurality of SNPs comprises some or all SNPs in a reference genome sequence of a species of the test subject with a minor allele frequency (MAF) of more than 15%. The plurality of SNPs can comprise or comprise about 11724 SNPs.

In some embodiments, the BAF value of the SNP is determined using absence of a label at the position of the SNP in the GM data generated from the control sample. In some embodiments, the BAF value of the SNP for each of the plurality of control samples is determined by: determining a signal strength of a B-allele of the SNP in the GM data generated from the control sample. The BAF value of the SNP for each of the plurality of control samples can be determined by: determining the BAF value of the SNP for the control sample using the signal strength of the B-allele of the SNP for the control sample. In some embodiments, determining the signal strength of the B-allele of the SNP in the GM data generated from the control sample comprises: determining the signal strength of the B-allele of the SNP in the GM data generated from the control sample using absence of a label at the position of the SNP in the GM data generated from the control sample. The signal strength of the B-allele of the SNP for the control sample can be a ratio of (i) a number of deoxyribonucleic acid (DNA) molecules comprising the SNP and without a label at the position of the SNP in the GM data generated from the control sample and (ii) a number of DNA molecules comprising the SNP in the GM data generated from the control sample.

In some embodiments, the processor is programmed by the executable instructions to perform: determining a separation between a pair of clusters of the plurality of clusters for a second SNP of the SNPs is below a separation threshold, optionally wherein the separation comprises a Silhouette score. The processor can be programmed by the executable instructions to perform: removing the second SNP from BAF value and normalized BAF value determination, and/or calculating a loss of heterozygosity (LOH) without using the second SNP, the BAF value of the second SNP, and/or the normalized BAF value of the second SNP. In some embodiments, the processor is programmed by the executable instructions to perform: calculating a loss of heterozygosity (LOH) using the normalized BAF values of two or more of the SNPs.

In some embodiments, a label is assigned to two reference label positions for at least a predetermined percentage of DNA molecules comprising a third SNP the SNPs. The processor can be programmed by the executable instructions to perform: removing the third SNP from BAF value and normalized BAF value determination. The processor can be programmed by the executable instructions to perform: calculating a loss of heterozygosity (LOH) without using the third SNP, the BAF value of the third SNP, and/or the normalized BAF value of the third SNP.

In some embodiments, the SNP is at a region with a copy number (CN) loss or a copy number gain. The SNP can be at a region with 0% loss of one copy. The SNP can be at a region with 50% loss of one copy. The SNP can be at a region with complete loss of one copy. The SNP can be at a region with 50% trisomy. The SNP can be at a region with complete trisomy. The SNP can be at a region with complete tetrasomy. Determining the BAF value of the SNP for each of the plurality of control samples can comprises: determining a signal strength of a B-allele of the SNP in the GM data generated from the control sample using a number of deoxyribonucleic acid (DNA) molecules comprising the SNP mapped to a loss map, a number of DNA molecules comprising the SNP mapped to all maps, and/or a number of DNA molecules mapped to a gain/duplicate map. Determining the BAF value of the SNP for each of the plurality of control samples can comprises: determining the BAF value of the SNP for the control sample using the signal strength of the B-allele of the SNP for the control sample.

In some embodiments, the plurality of clusters is generated from the BAF values of the SNP for control samples of the plurality of control samples using connectivity-based clustering, centroid-based clustering, distribution-based clustering, density-based clustering, grid-based clustering, or a combination thereof. In some embodiments, the plurality of clusters comprises three clusters representing AA, AB, and BB genotypes of the SNP, optionally wherein the three cluster centers of the three clusters representing AA, AB, and BB genotypes are at about 0, 0.5, and 1.0 respectively. The three cluster centers representing AA, AB, and BB may not be at 0, 0.5, and 1.0 respectively. In some embodiments, wherein the processor is programmed by the executable instructions to perform: determining a cluster center of each of the plurality of clusters. A cluster center of a cluster of the plurality of cluster can be an average, a mean, a median, or a combination thereof, of the BAF values in the cluster. In some embodiments, a cluster of the plurality of clusters representing BB genotype for a SNP comprises an insufficient number of BAF values. The cluster center of the cluster comprising an insufficient number of BAF values can comprise a measure of cluster centers representing BB genotypes for two or more of the SNPs with sufficient numbers of BAF values. The measure of cluster centers representing BB genotypes can be an average, a mean, a median, or a combination thereof, of the cluster centers representing BB genotypes.

In some embodiments, receiving the GM data generated from the test sample obtained from the test subject comprises: generating the GM data from the test sample obtained from the test subject. In some embodiments, the GM data generated from the test sample obtained from the test subject comprises a deoxyribonucleic acid (DNA) consensus map for the test subject, optionally wherein the DNA consensus map comprises presence and/or absence of labels at the position of the SNP.

In some embodiments, determining the BAF value of the SNP for the test sample comprises: determining the BAF value of the SNP using absence of a label at the position of the SNP in the GM data generated from the test sample. In some embodiments, determining the BAF value of the SNP for the test sample comprises: determining a signal strength of a B-allele of the SNP in the GM data generated from the test sample. Determining the BAF value of the SNP for the test sample can comprise: determining the BAF value of the SNP for the test sample using the signal strength of the B-allele of the SNP for the test sample. In some embodiments, determining the signal strength of the B-allele of the SNP in the GM data generated from the test sample comprises: determining the signal strength of the B-allele of the SNP in the GM data generated from the test sample using absence of a label at the position of the SNP in the GM data generated from the test sample. The signal strength of the B-allele of the SNP for the test sample can be a ratio of (i) a number of deoxyribonucleic acid (DNA) molecules comprising the SNP and without a label at the position of the SNP in the GM data generated from the test sample and (ii) a number of DNA molecules comprising the SNP in the GM data generated from the test sample.

In some embodiments, determining the normalized BAF value for the test sample comprises: determining the normalized BAF value of the SNP for the test sample from the BAF value of the SNP for the test sample using two cluster centers of the plurality of cluster centers, or cluster centers of two cluster centers of the plurality of cluster centers. The BAF value of the SNP can be smaller than the cluster center of the cluster representing AB genotype. The normalized BAF value of the SNP can be a ratio of (i) a distance between the cluster center of the cluster representing AA genotype and the BAF value of the SNP, and (ii) two times a distance between the cluster center of the cluster representing AA genotype and between the cluster center of the cluster representing AB genotype. The BAF value of the SNP can be greater than the cluster center of the cluster representing AB genotype. The normalized BAF value of the SNP can be one minus a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

In some embodiments, corresponding clusters of the pluralities of clusters represent a genotype. The processor can be programmed by the executable instructions to perform: clustering BAF values of SNPs for the test sample into a plurality of test sample clusters, representing the genotypes, each comprising a test sample cluster center. The processor can be programmed by the executable instructions to perform: determining a measure of the cluster centers of the clusters representing each of the genotypes, optionally wherein the measure of the cluster centers is an average, a mean, a median, or a combination thereof, of the cluster centers. The processor can be programmed by the executable instructions to perform: determining a difference between a test sample cluster center and the measure of the cluster centers of the clusters representing an identical genotype. The processor can be programmed by the executable instructions to perform: for a SNP of the SNPs, adjusting a cluster center of the cluster of the plurality of clusters for the SNP based on the difference to determine an adjusted cluster center. Determining the normalized BAF value of the SNP for the test sample can comprise: determining the normalized BAF value of the SNP for the test sample from the BAF value of the SNP for the test sample using one or more of the plurality of adjusted cluster centers. In some embodiments, the plurality of test sample clusters comprises three test sample clusters representing AA, AB, and BB genotypes of the SNP.

In some embodiments, the processor is programmed by the executable instructions to perform: creating a file or a report and/or generating a user interface (UI) comprising a UI element representing or comprising (i) the BAF value of the SNP for one, one or more, or each, of the plurality of control samples, (ii) the signal strength of the B-allele of the SNP for one, one or more, or each, of the plurality of control samples, (iii) the BAF value of the SNP for the test sample, (iv) the signal strength of the B-allele of the SNP for the test sample, and/or (v) the normalized BAF value of the SNP for the test sample. In some embodiments, wherein the sample comprises cells, cell-free DNA, cell-free fetal DNA, amniotic fluid, a blood sample, a bone marrow sample, a biopsy sample, or a combination thereof.

Also disclosed herein include a non-transitory computer-readable medium storing executable instructions, when executed by a system (e.g., a computing system), causes the system to perform any method or one or more steps of any method disclosed herein.

Details of one or more implementations of the subject matter described in this specification are set forth in the accompanying drawings and the description below. Other features, aspects, and advantages will become apparent from the description, the drawings, and the claims. Neither this summary nor the following detailed description purports to define or limit the scope of the inventive subject matter.

### BRIEF DESCRIPTION OF THE DRAWINGS

FIG. 1 shows a non-limiting exemplary illustration of BAF values calculated for a single SNP across a population of samples.

FIG. 2 shows a non-limiting exemplary illustration of BAF values for a single SNP across a population of samples grouped into three clusters. Cluster centers (e.g., median values) are represented by plus signs.

FIG. 3 shows a non-limiting exemplary illustration of determining the (normalized) BAF value of a SNP (the same as the one illustrated in FIGS. 1-2) of a new sample.

FIG. 4 shows a non-limiting exemplary illustration of determining the (normalized) BAF value of a SNP (the same as the one illustrated in FIGS. 1-2) of a new sample.

FIG. 5 illustrates a non-limiting exemplary workflow of OGM.

FIG. 6 is a flow diagram showing an exemplary method for determining a BAF value (or BAF values), such as a normalized BAF value, from GM data.

FIG. 7 is a block diagram of an illustrative computing system configured to implement determining a BAF value (or BAF values), such as a normalized BAF value, from GM data.

Throughout the drawings, reference numbers may be re-used to indicate correspondence between referenced elements. The drawings are provided to illustrate example embodiments described herein and are not intended to limit the scope of the disclosure.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments can be utilized, and other changes can be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods for determining a B-allele frequency (BAF) value (e.g., a normalized BAF value) from genome mapping (GM) data. In some embodiments, a method for determining a normalized BAF value from GM data is under control of a processor (e.g., a hardware processor or a virtual processor) and comprises: receiving genome mapping (GM) data generated from a plurality of control samples obtained from a plurality of control subjects. The method can comprise: determining a B-allele frequency (BAF) value of a single nucleotide polymorphism (SNP) of a gene (or a SNP in or relative to a reference genome sequence) for each of the plurality of control samples using the GM data generated from control sample. The method can comprise: clustering the BAF values of the SNP of the gene for control samples of the plurality of control samples into a plurality of clusters each comprising (or has or is associated with) a cluster center. The method can comprise: receiving GM data generated from a test sample obtained from a test subject. The method can comprise: determining a BAF value of the SNP of the gene for the test sample using the GM data of the test sample. The method can comprise: determining a normalized BAF value of the SNP of the gene for the test sample from the BAF value of the SNP of the gene for the test sample using one or more (e.g., 2, 3, 4, 5, or more) of the cluster centers (or using values of one or more of the cluster centers). In some embodiments, the GM data comprises optical genome mapping (OGM) data. In some embodiments, the GM data comprises electronic genome mapping (EGM) data.

Disclosed herein include methods for determining a B-allele frequency (BAF) value (e.g., a normalized BAF value) from genome mapping (GM) data. In some embodiments, a method for determining a normalized BAF value from GM data is under control of a processor (or a hardware processor or a virtual processor) and comprises: receiving genome mapping (GM) data generated from a plurality of control samples obtained from a plurality of control subjects. The method can comprise: determining a B-allele frequency (BAF) value of each single nucleotide polymorphism (SNP) of SNPs of a plurality of SNPs (e.g., SNPs in or relative to a reference genome sequence) for each of the plurality of control samples using the GM data generated from control sample. The method can comprise: clustering the BAF values of the SNP for control samples of the plurality of control samples into a plurality of clusters each comprising a cluster center. The method can comprise: receiving GM data generated from a test sample obtained from a test subject. The method can comprise: determining a BAF value of the SNP for the test sample using the GM data of the test sample. The method can comprise: determining a normalized BAF value of the SNP for the test sample from the BAF value of the SNP for the test sample using one or more (e.g., 2, 3, 4, 5, or more) of the cluster centers (or values of one or more of the cluster centers). In some embodiments, the GM data comprises optical genome mapping (OGM) data. In some embodiments, the GM data comprises electronic genome mapping (EGM) data.

Disclosed herein include systems for determining a B-allele frequency (BAF) value, or a normalized BAF value, from genome mapping (GM) data. In some embodiments, a system for determining a normalized BAF value from GM data comprises non-transitory memory configured to store: executable instructions. The non-transitory memory can be configured to store: genome mapping (GM) data generated from a plurality of control samples obtained from a plurality of control subjects. The non-transitory memory can be configured to store: a B-allele frequency (BAF) value of a single nucleotide polymorphism (SNP) of a gene (or in a reference genome sequence) for each of the plurality of control samples determined using the GM data generated from control sample. The non-transitory memory can be configured to store: a plurality of clusters, each comprising a cluster center, generated from BAF values of the SNP of the gene for control samples of the plurality of control samples. The system can comprise: a processor (e.g., a hardware processor or a virtual processor) in communication with the non-transitory memory. The processor can be programmed by the executable instructions to perform: receiving GM data generated from a test sample obtained from a test subject. The processor can be programmed by the executable instructions to perform: determining a BAF value of the SNP of the gene for the test sample using the GM data of the test sample. The processor can be programmed by the executable instructions to perform: determining a normalized BAF value of the SNP of the gene for the test sample from the BAF value of the SNP of the gene for the test sample using one or more of the cluster centers (or values of one or more of the cluster centers). In some embodiments, the GM data comprises optical genome mapping (OGM) data. In some embodiments, the GM data comprises electronic genome mapping (EGM) data.

Disclosed herein include systems for determining a B-allele frequency (BAF) value (e.g., a normalized BAF value) from genome mapping (GM) data. In some embodiments, a system for determining a BAF value from GM data comprises: non-transitory memory configured to store: executable instructions. The non-transitory memory can be configured to store: genome mapping (GM) data generated from a plurality of control samples obtained from a plurality of control subjects. The non-transitory memory can be configured to store: a B-allele frequency (BAF) value of each single nucleotide polymorphism (SNP) of SNPs of a plurality of SNPs (e.g., a plurality of SNPs in a reference genome sequence) for each of the plurality of control samples determined using the GM data generated from control sample. The non-transitory memory can be configured to store: a plurality of clusters, each comprising a cluster center, generated from BAF values of the SNP for control samples of the plurality of control samples. The system can comprise: a processor (e.g., a hardware processor or a virtual processor) in communication with the non-transitory memory. The processor can be programmed by the executable instructions to perform: clustering BAF values of the SNP for control samples of the plurality of control samples into a plurality of clusters each comprising a cluster center. The processor can be programmed by the executable instructions to perform: receiving GM data generated from a test sample obtained from a test subject. The processor can be programmed by the executable instructions to perform: determining a BAF value of the SNP for the test sample using the GM data of the test sample. The processor can be programmed by the executable instructions to perform: determining a normalized BAF value of the SNP for the test sample from the BAF value of the SNP for the test sample using one or more of the plurality of cluster centers. In some embodiments, the GM data comprises optical genome mapping (OGM) data. In some embodiments, the GM data comprises electronic genome mapping (EGM) data.

Also disclosed herein include a non-transitory computer-readable medium storing executable instructions, when executed by a system (e.g., a computing system), causes the system to perform any method or one or more steps of any method disclosed herein.

### Determining BAF Values

Disclosed herein include systems, devices, and methods for obtaining from GM (e.g., optical genome mapping (OGM) or electronic genome mapping (EGM) data) B-allele frequency (BAF) values (or values similar to BAF) that are generated by SNP arrays. Before getting to the actual clustering part for optimizing BAF values, the strength of A and B alleles from GM data need to be calculated. Two different approaches are described below. One of the two approaches can be used, or both can be used together or separately.

First Approach. The labels used in GM attach to a specific sequence in the genome (typically to a six letter sequence). If there are any SNPs in any of the six letters at each binding location, this will cause a lack of florescence at that point. A list of known SNPs with Minor Allele Frequency (MAF) greater than, for example, 15% can be determined. This list can be intersected with the list of all binding location in the genome to generate, for example, a list of 11724 positions (in hg38) that can be used to calculate BAF values. In some embodiments, lower MAF frequency (e.g., 5%) can be used. With a lower MAF value, there are more locations that will be mostly homozygous. To calculate the BAF values (e.g., unnormalized) BAF values, at each of these approximately 11000 positions (or more or fewer locations depending on the MAF frequency used), the number of missing labels can be counted (or determined). The number of missing labels can be the B-allele signal. The number of missing labels (the B-allele signal) can be divided by total the number of molecules (DNA molecules) at that position to calculate (or determine) the BAF value.

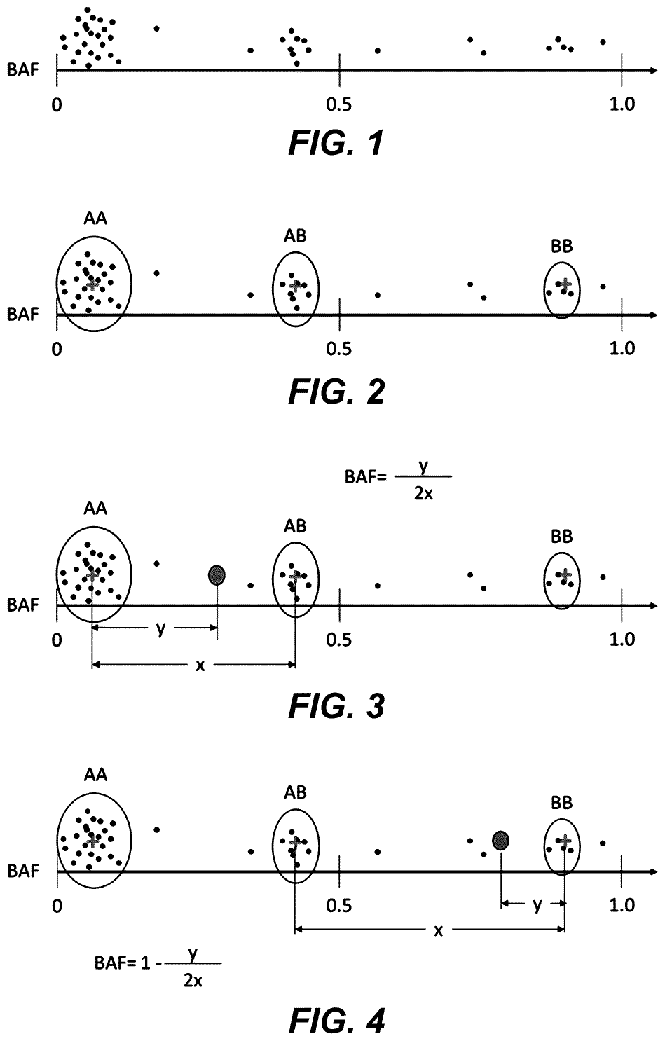

The above approach can provide signal strength for the B-allele. Clustering can be used to adjust for specific SNP behavior. For example, each SNP across a large set of “control” samples can be looked at. Imagine looking at the BAF values calculated for a single SNP across a population of samples and plotting the BAF values as calculated based on the approach above for this single SNP. These BAF values can be plotted on a single axis as illustrated in FIG. 1 (the Y-axis has no real meaning here and just to help visualize).

Since most of the SNPs are not very frequent in the population, most BAF values get clustered around 0 with a few somewhere in the middle and even fewer closer to 1, for example. As FIG. 1 shows, these BAF values might not be exactly at 0, 0.5, and 1.0. The next step would be to “cluster” this data (these BAF values) into three groups representing AA, AB, and BB (which can be genotypes) and then identifying a cluster center (e.g., the median value) in each cluster (represented as plus signs in FIG. 2).

In some implementations, cluster-based corrections may be performed on the fraction of molecules that are labeled at each SNP position, where the most common case is for the label to not be disrupted by the SNP, with labeling fractions slightly less than one 1.0. The labeling fractions may be converted to BAF values by alternately flipping each value to 1-value.

The clustering of the SNPs can be done using a number of different methods, including Gaussian Mixture Model (GMM). As shown in the plots of FIGS. 1-2, there will be samples for a SNP where BAF values fall outside any cluster, which is fine. In this process, if it is not possible to reliably identify any clusters separating the three groups, this SNP position can be marked as one that is too noisy to use and removed from usage in loss of heterozygosity (LOH) calculation in some implementations. To evaluate the quality of the clusters, the separation of the cluster centers from each other can be looked at. A minimum separation may be required. A score like the Silhouette score can be used for evaluating the clusters.

In some instances, it is possible that the BB cluster might have too few samples (or no samples) to cluster. This should not cause the SNP position to be removed. A constant value can be used for the cluster center (or cluster median) there, for example, by using median of all SNPs for the BB clusters.

Once the cluster centers are identified, a normalized BAF value for this SNP position for a new sample (e.g., a test sample) can be generated. For example, the formula below as illustrated in FIG. 3 can be used if the original (unnormalized) BAF value for this SNP position for the new sample is between AA and AB:

$$( normalized ) BAF = \frac{y}{2 x}$$

where y is the distance between the center of the AA cluster (e.g., AA median) and the original (unnormalized) BAF value for this SNP position for the new sample.

If the original (unnormalized) BAF value for this SNP position for the new sample is greater than the center of the AB cluster (e.g., AB median), the formula below as illustrated in FIG. 4.

$$( normalized ) B A F = 1 - \frac{y}{2 x}$$

If the unnormalized (or non-normalized) value of the BAF is less than the center of the AA cluster (e.g., AA median), the normalized value can be set to zero. If the unnormalized (or non-normalized) value of the BAF is greater than the center of the BB cluster (e.g., BB median), the normalized value can be set to zero.

Additional enhancements can be added in some embodiments the clustering correction described above, such as dropping SNP positions with too many split label mappings and a per-sample correction to accounts for differences in labeling efficiency.

Split-Label Enhancement. GM label positions that are too close together in the reference genome can appear as a single label position in a subset of sample molecules. In these cases, the single label position in the sample molecules can be assigned a split mapping that maps it to both reference label positions. This causes the SNP labeling disruption to be more complex. In some embodiments, the BAF values calculated at these positions are discarded. In some embodiments, if more than a certain percentage (e.g., about 10%) of the sample molecules have split label mappings at a particular SNP position, then the SNP position results can be discarded. The BAF data overall is cleaner with such SNP position results discarded.

Adjustment. Correction for per-sample differences in labeling efficiency can be performed. The cluster-based corrections (or adjustments or optimizations) for a new sample for each SNP position can be adjusted using a set of three per-sample constant offsets. For example, consider a case where the median across all cluster correction centers for the homozygous-reference label-fractions is 0.93 (or 0.07), but then, for the sample, the center of the cluster of uncorrected homozygous-reference label-fractions is 0.95 (0.05). In this case, each cluster correction center can be adjusted up by a constant offset of +0.02=0.95−0.93 (or −0.02=0.05−0.07) before using the cluster correction center to correct the individual SNP position. The centers of the three sample clusters (homozygous-reference, heterozygous, homozygous-alternate) for the uncorrected label fractions can be calculated efficiently using K-means, in only a single dimension.

Second Approach. The insertions and deletions detected through the structural variation (SV) pipeline can be used to estimate BAF for all markers in the region of gain/loss. This approach may not be applicable to other SV types (e.g., inversion, translocation, etc.) that do not impact the copy number because the need to represent the BAF for a “region” of the genome that has an allelic imbalance. To arrive at the BAF values, two different methods can be used depending on the event type.

For a CN loss region, for each label position in the loss region, the following can be counted or calculated:
X=Number of molecules mapped to the loss map
Y=Total number of molecules at that point (labels mapped to all maps)
   Strength of B-Allele can be calculated as

$$α = \frac{2 X}{Y + X}$$

$$Then$$

$$BAF = \frac{1 - α}{2 - α}$$

For a CN gain region, for each label position that is mapped to the reference map and another map with a tandem duplication, the following can be counted or calculated:
Z=Number of molecules mapped to the gain/duplicate map
X=Z/2
Y=Number of molecules mapped to the reference map+Z/2
   Strength of B-Allele can be calculated as

$$α = \frac{2 X}{Y}$$

$$Then$$

$$BAF = \frac{1 + α}{2 + α}$$

A few examples on how the above works are illustrated below for loss events and gain events.

### Example 1—Loss Event: Complete Loss of One Copy

X=50 and Y=50 (half the molecules representing one allele are mapped to loss map and the other half are to the reference)

$$α = \frac{2 ( 50 )}{100} = 1 ; B A F = \frac{1 - 1}{2 - 1} = 0 ,$$

### Example 2—Loss Event: 50% Loss of One Copy

X=25 and Y=75

$$α = \frac{2 ( 25 )}{100} = 0.5 ; B A F = \frac{1 - 0.5}{2 - 0.5} = 0.33 ,$$

which is the expected value

### Example 3—Loss Event: 0% Loss of One Copy (at Limit)

X=0 and Y=100

$$α = \frac{2 ( 0 )}{100} = 0 ; B A F = \frac{1 - 0}{2 - 0} = 0.5 ,$$

which is the expected value

For more than one copy number loss, the BAF is undefined so negative values should just leave no BAF values in the result (N/A).

### Example 4—Gain Event: Complete Trisomy

X=100 and Y=200 (an amount equal to half of the molecules mapped to the reference map representing one additional allele are mapped to duplication)

$$α = \frac{2 ( 100 )}{200} = 1 ; B A F = \frac{1 + 1}{2 + 1} = 0.67 ,$$

which is the expected value

### Example 5—Gain Event: 50% Trisomy

X=50 and Y=200

$$α = \frac{2 ( 50 )}{200} = 0.5 ; B A F = \frac{1 + 0.5}{2 + 0.5} = 0.6 ,$$

which is the expected value

### Example 6—Gain Event: Complete Tetrasomy

X=200 and Y=200 (this assumes it is not a balanced duplication and only one allele is gained twice)

$$α = \frac{2 ( 200 )}{200} = 2 ; B A F = \frac{1 + 2}{2 + 2} = 0.75 ,$$

which is the expected value

The above schemes create a non-normalized “first approximation” to the BAF values. The above can be optimized by performing clustering approach by looking at behavior of labels across a large number of samples (e.g., 50-100) as described herein.

### Optical Genome Mapping

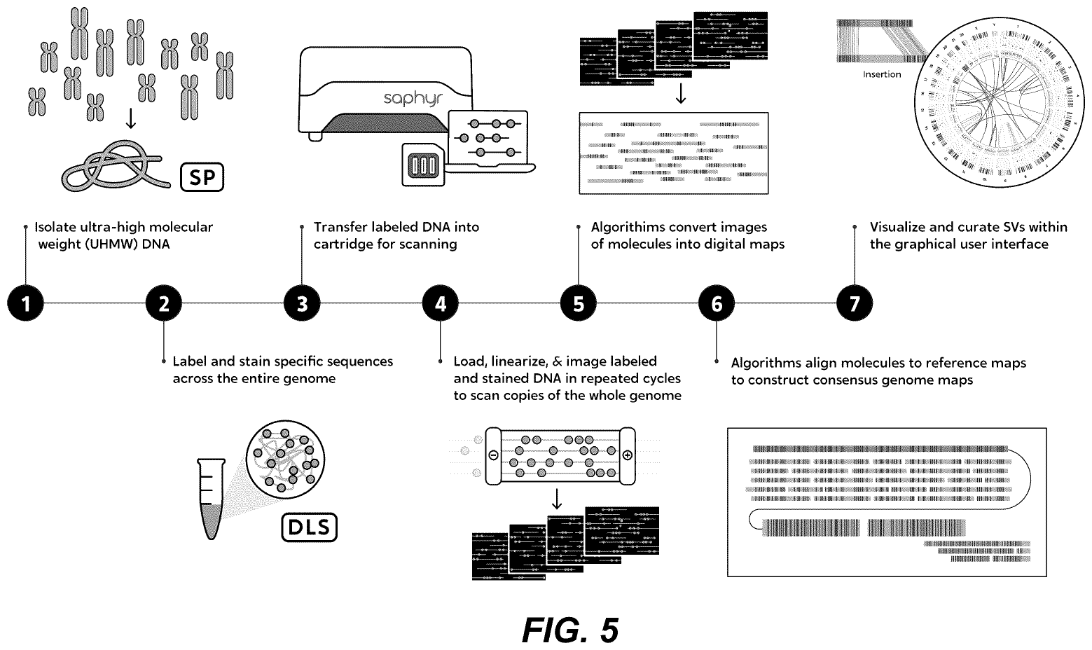

FIG. 5 illustrates a non-limiting exemplary workflow of optical genome mapping (OGM). The data generated from OGM can be used to determine BAF values of SNPs for a sample. The OGM workflow can start with mega-base size DNA isolation, e.g., 150 kbp or longer. A single enzymatic reaction can label the genome at a specific sequence motif occurring, e.g., approximately 15 times per 100 kbp in the human genome. The long, labeled DNA molecules can be linearized in nanochannel arrays (e.g., provided by a cartridge or chip, such as a Saphyr Chip®, Bionano Genomics, Inc. (San Diego, CA)) and imaged in an automated manner by an OGM instrument (e.g., Saphyr® System, Bionano Genomics, Inc. (San Diego, CA)). The molecules can be assembled into local maps or whole genome maps. Changes in patterning or spacing of the labels can be detected, genome-wide, to call structural variants.

Optical Genome Mapping (OGM) is an imaging technology which evaluates the fluorescent labeling pattern of individual DNA molecules to perform an unbiased assessment of genome-wide structural variants down to, e.g., 500 base pairs (bp) in size, a resolution that far exceeds conventional cytogenetic approaches. OGM can rely on a specifically designed extraction protocol facilitating the isolation of high molecular weight (HMW) or ultra-high molecular weight (UHMW) DNA ultra-high molecular weight (UHMW) DNA. This protocol can, in some embodiments, utilize a paramagnetic disk purposed with trapping DNA for wash steps thereby reducing sheering forces present in standard column-based extraction methods. The result can be DNA fragments (or molecules) of about 150 kilobases (kbp) to megabases (Mbp) in size, about 5-10× longer than the average fragment size from conventional DNA isolations techniques. Referring to FIG. 5, DNA can be fluorescently labeled via covalent modification at a motif (which can be 4, 5, 6, 7, 8, 9, 10, or more nucleotides in length), such as a hexamer motif (e.g., the CTTAAG hexamer motif), generating genome-wide density of a number of labels per 100 kb in sequence specific patterns (e.g., approximately 14-17 labels per 100 kb, or 10, 11, 12, 13, 14, 15, 16, 17, 18, 19, 20, or more or fewer labels per 100 kb). Labeled DNA can be loaded on chips (e.g., silicon chips) composed of hundreds of thousands of parallel nanochannels where individual DNA molecules are linearized, imaged, and digitized. The specific labeling profile of individual DNA molecules, including spacing and pattern of hexamers labels, can be subsequently grouped based on similarity, producing about 500 kbp (or longer or shorter, such as 300 kbp, 400 kbp, 500 kbp, 600 kbp, 700 kbp, 800 kbp, 900 kbp, 1000 kbp) to megabase-sized consensus maps, which can be compared in silico to the expected labeling pattern of a reference genome (FIG. 5). This imaging technology converts DNA into a “barcode” whose labeling profile and characteristics can sensitively and specifically resolve copy number and structural variation without the need for sequence level data (FIG. 5). The quality of the DNA, including both size and labeling characteristics, as well as the number of images captured can influence genome-wide coverage. For example, each flow cell, which can accommodate a single specimen, can generate, for example, up to 5000 Gigabase pairs (Gbp) of raw data (or 3000 Gbp, 4000 Gbp, 5000 Gbp, 6000 Gbp, 7000 Gbp, 8000 Gbp, 9000 Gbp, 10000 Gbp, or more or less, of raw data), achieving a maximum theoretical genome-wide coverage of about 1250× (or 500×, 750×, 1000×, 1250×, 1500×, 1750×, 2000×, or more or less). Bioinformatics analyses can be performed. Example bioinformatics analysis can include: de novo structural variant analysis for typical germline assessments (e.g., greater than about 80×-coverage; requiring greater than about 400 Gbp data collection) or ‘Rare Variant Analysis (RVP)’ for somatic assessment down to a ˜5% variant allele fraction (e.g., greater than about 340× coverage; requiring greater than about 1500 Gbp data). Both algorithms facilitate the detection of a wide array of structural variants; from copy number gains/losses to balanced/unbalanced translocations and insertions to inversions.

Optical genome mapping (OGM) can be used to analyze large eukaryotic genomes and their structural features at a high resolution. OGM uses linearized strands of high molecular weight (HMW) or ultra-high molecular weight (UHMW) DNA that are far longer than the DNA sequences analyzed in current second- and third-generation sequencing methods, achieving average read lengths in excess of 200 kbp. The usage of long molecules in OGM can allow repetitive regions and other regions that are complicated to map to be spanned more easily than with short molecules. This leads to the creation of maps that may cover the whole arm of a chromosome and yet allow the detection of insertions and deletions as small as 500 bp (or longer or shorter, such as 300 kbp, 400 kbp, 500 kbp, 600 kbp, 700 kbp, 800 kbp, 900 kbp, 1000 kbp) other SVs may need to be 30 kbp (or 10 kbp, 20 k kbp, 30 kbp, 40 kbp, or 50 kbp)) or larger to be detectable. OGM can be used to, for example, detect the breakpoints of chromosomal translocations, for the diagnosis of facioscapulohumeral muscular dystrophy (FSHD). OGM may be used as a cytogenomic tool for prenatal diagnostics

Extraction/Isolation. UHMW DNA can be extracted for OGM, for example. UHMW DNA extraction can be done using isolation kits, such as kits from Bionano Genomics, Inc. (San Diego, CA). In some embodiments, DNA from approximately 1.5×10⁶ cells (or 1× 10⁵, 1.5×10⁵, 2.5×10⁵, 5× 10⁵, 7.5×10⁵, 1×10⁶, 1.5×10⁶, 2.5×10⁶, 5×10⁶, 7.5×10⁶, 1×10⁷ or more or fewer cells) can be extracted. The extraction can include immobilizing cells in agarose plugs and lysing the immunized cells by proteinase K; thereafter. The extraction can include washing, recovering, and quantifying the genomic DNA. Alternatively or additionally, the genomic DNA can be bound to a magnetic disk. Subsequently, the DNA can be washed, recovered, and quantified.

Labeling and Processing. A sufficient quantity of UHMW DNA (e.g., 250 ng, 500 ng, 750 ng, 1000 ng, 1250 ng, 1500 ng, 1750 ng, 2000 ng, or more UHMW DNA) can be labeled with a fluorophore. Such labeling can be done using a methyltransferase, such as the methyltransferase direct labeling enzyme (DLE-1) at the recognition motif of the methyltransferase, such as CTTAAG. This can generate a number of labels per 100 kbp (e.g., approximately 14-15 labels per 100 kbp, or 10, 11, 12, 13, 14, 15, 16, 17, 18, 19, 20, or more or less labels per kbp) when labeling human genomic DNA. In some embodiments, such labeling can be done using another enzyme (e.g., an endonuclease) at the recognition motif of the enzyme (e.g., GCTCTTCN of endonuclease Nt.BspQI).

Thereafter, the DNA can be dialyzed, its backbone stained, and finally the prepared DNA can be applied to flow cells (e.g., G1.2 flow cells from Bionano Genomics, Inc.) The flow cell can then be inserted into an OGM instrument, such as the Saphyr® instrument from Bionano Genomics, Inc. In the instrument, the DNA can be fed by electrophoresis into the nanochannels of the flow cell for linearization. DNA-filled nanochannels can be scanned using, for example, a fluorescence microscope. The captured images can be converted to electronic representations of the DNA molecules. The virtual DNA strands can then filtered and de novo assembled into maps (FIG. 5).

OGM Data Assembly. The data acquired with the OGM instrument can be processed. For example, the raw data can be filtered for a minimum length of 150 kbp (or 100 kbp, 125 kbp, 150 kbp, 175 kbp, 200 kbp, or more) and minimum of nine labels (or 5, 6, 7, 8, 9, 10, 11, 12, 13, 14, 15, or more labels) per molecule (or fragment). The filtered molecules can be assembled, e.g., with de novo assembly. The consensus maps of the molecules can be aligned to a reference genome sequence, such as the human reference genome GRCh38. Variants can be detected. Variants detection can be performed using, for example, a SV pipeline, comparing the maps to the aligned reference genome. There, patterns of markers from the maps deviating from the reference become apparent. Variants detections can be performed using, for example, a CNV pipeline,” which quantifies the mapped molecules and hence is able to detect gains and losses of several hundred kbp in size.

The results of the SV pipeline can then be augmented by, for example, a variant annotation pipeline, which adds quality metrics for the called variants and supplies their estimated frequency in the human population based on an internal database. The optional step of filtering based on the frequency of the SVs in the internal database may (or may not) be used in some implementations. The SVs can be detected or called. Automatic calling can be based on the confidence scores and sizes of the SVs (insertions and deletions: confidence>0, size>500 bp; inversions: confidence>0.7, size>30 kbp; duplications: confidence=−1, size>30 kbp; intrachromosomal translocations: confidence>0.3; interchromosomal translocations: confidence>0.65; CNV confidence>0.99, size>500 kbp). Additionally, each called SV can be required to be spanned by >5 strands of DNA.

The total amount of unfiltered DNA scanned by the OGM system can be, or be about, 750 Gbp, 800 Gbp, 850 Gbp, 900 Gbp, 916 Gbp, 925 Gbp, 950 Gbp, 1000 Gbp, 1250 Gbp, or more, per sample on average. An effective coverage of the reference can be, or can be greater than, 40×, 50×, 60×, 70×, 80×, 90×, or more, per sample. The effective coverage of the reference can be defined as the total length of filtered (≥150 kbp) and aligned molecules divided by the length of the reference genome after de novo assembly

Further details regarding various aspects of OGM can be found in U.S. Pat. Nos. 11,359,244; 11,292,713; 11,291,999; 10,995,364; 10,844,424; 10,676,352; 10,669,586; 10,654,715; 10,435,739; 10,247,700; 10,000,804; 10,000,803; 9,845,238; 9,809,855; 9,804,122; 9,725,315; 9,536,041; 9,533,879; 9,310,376; 9,181,578; 9,061,901; 8,722,327; and 8,628,919; as well as published PCT Application Publication Nos. WO2020/005846; WO2016/036647; WO2015/134785; WO2015/130696; WO2015/126840; WO2015/017801; WO2014/200926; WO2014/130589; WO2014/123822; WO2013/036860; WO2012/054735; WO2011/050147; WO2011/038327 and WO2010/13532; the content of each of which is incorporated herein by reference in its entirety.

### Electronic Genome Mapping

For electronic genome mapping (EGM), high molecular weight (HMW) or ultra-high molecular weight (UHMW) DNA molecules (e.g., 50 kbp to 500 kbp) can be isolated from a sample (e.g., a cell sample, a blood sample). The isolated DNA molecules can be labelled at known recognition sites. Labeling can include DNA nicking translocation and label (or tag) insertion. The recognition sites can be, for example, 4 kbp apart on average. A DNA binding protein (e.g., RecA) can be used to stiffen the DNA molecules. An EGM chip (also referred to as an EGM detector) can comprise solid-state nanochannels (e.g., 256 parallel nanochannels), each with its own electronic sensor. The EGM chip can be in an EGM instrument. The labeled DNA molecules can be injected into the EGM chip. Single DNA molecules can be electrophoretically moved through a nanochannel. Single DNA molecules can be electrophoretically moved through nanochannels at the same time. In a nanochannel, the labels (or tags) of a DNA molecule can be electronically detected by changes in resistance, which can be inferred from changes in voltage. When a DNA molecule enters the nanochannel, it blocks the current that can go through the channel and can be measured as a voltage change. When a label (or tag) is also present on the DNA molecule, the current is further reduced resulting in a sharp signal. The voltage can be measured as a function of time so the time that a nanochannel is empty, the time it is occupied by untagged DNA, and the time each label (or tag) goes through the nanochannel can be determined. The times between voltage peaks correspond to distances between labels on a DNA molecule. These times can be converted to distances for each DNA molecule. The results can include single molecule maps with the location of each label (or tag). The single molecule maps for a single sample can be assembled into local maps or a whole genome map of all labeled (or tagged) locations for the sample which can be aligned against a reference genome. Analysis such as structural variant (SV) analysis can be performed.

### Example BAF Value Determination

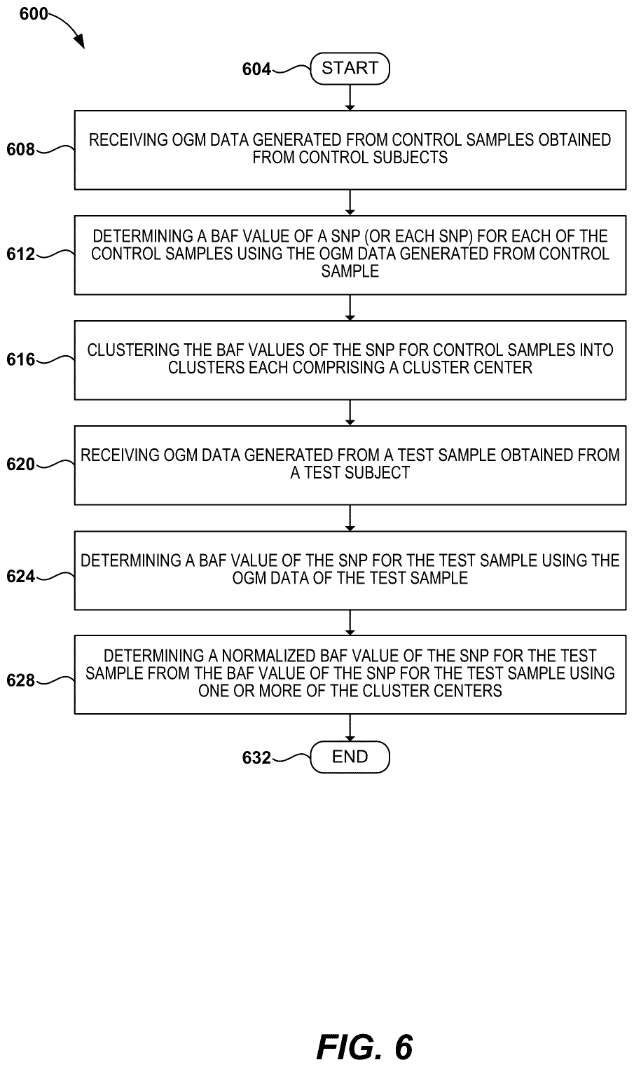

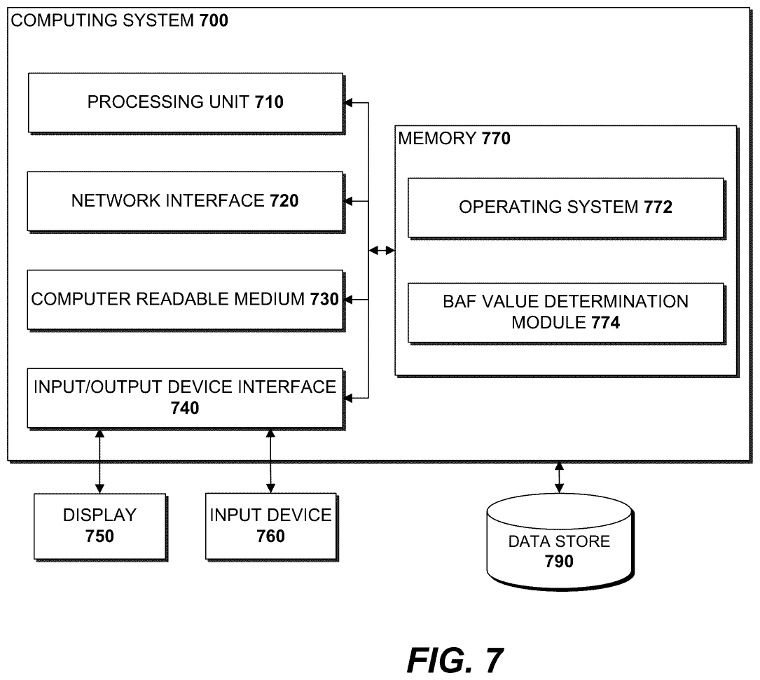

FIG. 6 is a flow diagram showing an exemplary method 600 of determining a BAF value (e.g., a normalized BAF value) from genome mapping (GM) data. The GM data can comprise optical genome mapping (OGM) data. Alternatively or additionally, the GM data can comprise electronic genome mapping (EGM) data. The method 600 may be embodied in a set of executable program instructions stored on a computer-readable medium, such as one or more disk drives, of a computing system. For example, the computing system 700 shown in FIG. 7 and described in greater detail below can execute a set of executable program instructions to implement the method 600. When the method 600 is initiated, the executable program instructions can be loaded into memory, such as RAM, and executed by one or more processors of the computing system 700. Although the method 600 is described with respect to the computing system 700 shown in FIG. 7, the description is illustrative only and is not intended to be limiting. In some embodiments, the method 600 or portions thereof may be performed serially or in parallel by multiple computing systems.

After the method 600 begins at block 604, the method 600 proceeds to block 608, where a computing system (such as the computing system 700) receives GM data generated from a plurality of control samples obtained from a plurality of control subjects. Each control sample can be obtained from a different control subject. Two control samples can be obtained from one control subject. The number of the control samples can be, for example, 10, 20, 30, 40, 50, 60, 70, 80, 90, 100, 200, 300, 400, 500, 750, 1000, 1500, 2500, 5000, 7500, 10000, or more. The number of the control subjects can be, for example, 20, 30, 40, 50, 60, 70, 80, 90, 100, 200, 300, 400, 500, 750, 1000, 1500, 2500, 5000, 7500, 10000, or more.

In some embodiments, the GM data generated from a control sample obtained from a control subject can comprise a deoxyribonucleic acid (DNA) consensus map for the control subject. The DNA consensus map can comprise presence and/or absence of labels (or signals) at (or corresponding to, from, or mapped to) the position of the SNP. For EGM, signals can be electric signals. The presence of a label (which can be a non-fluorescent label) can result in a change in the electric signal. For OGM, labels can be fluorescent labels, and signals can be fluorescent signals. For example, the DNA consensus map can comprise presence and/or absence of fluorescent labels (or fluorescent signals) at (or corresponding to, from, or mapped to) the position of the SNP.

The method 600 proceeds from block 608 to block 612, where the computing system determines a BAF value (an unnormalized or non-normalized BAF value) of a single nucleotide polymorphism (SNP) of a gene, or a SNP in a reference genome sequence, for each of the plurality of control samples using the GM data generated from control sample. In some embodiments, the computing system determines a BAF value of each SNP of SNPs of a plurality of SNPs for each of the plurality of control samples using the GM data generated from control sample. A BAF value can be or be about 0, 0.05, 0.1, 0.2, 0.3, 0.4, 0.5, 0.6, 0.7, 0.8, 0.9, 0.95, or 1 (e.g., on a scale of 0 to 1 where AA, AB, and BB genotypes have BAF values of 0, 0.5, and 1 respectively in an idealized situation).

A label for GM (e.g., a fluorescent label for OGM, or a label that is not fluorescent for EGM) can be attached to a predetermined sequence. The gene (or a reference genome sequence) can comprise the predetermined sequence. The SNP can be present at a position (e.g., position 1, 2, 3, 4, 5, 6, 7, 8, 9, or 10) in the predetermined sequence in the gene (or a reference genome sequence). The SNP can overlap the predetermined sequence in the gene (or a reference genome sequence). The nucleobase at the position in the predetermined sequence corresponds to (or is) an A-allele of the SNP. The presence of the label at the SNP in the GM data indicates an A-allele of the SNP. The absence of the label at the SNP in the GM data indicates a B-allele of the SNP. The predetermine sequence can six nucleotides (or 5, 6, 7, 8, 9, 10, or more nucleotides) in length. The predetermine sequence can comprise 5′-CTTAAG-3′. The predetermined sequence can be a recognition sequence of a methyltransferase. The methyltransferase can be a direct labeling enzyme (DLE-1).

The computing system can determine the plurality of SNPs. A SNP can overlap the predetermined sequence. Each of the plurality of SNPs can overlap the predetermined sequence. The plurality of SNPs can comprise one, some, or all SNPs present in a reference genome sequence that overlap the predetermined sequence. The species of the test subject and a species of a control subject (or each control subject) can be identical. The reference genome sequence can be that of a species (e.g., a vertebrate, a mammal, or a human) of the test subject (or a control subject), such as a reference human genome sequence (e.g., hg38 (GRCh38), hg19 (GRCh37), hg18, hg17, hg16). The plurality of SNPs can comprise some or all SNPs in a reference genome sequence with a minor allele frequency (MAF) of more than a predetermined percentage threshold, such as 15% (or 5%, 6%, 7%, 8%, 9%, 10%, 11%, 12%, 13%, 14%, 15%, 16%, 17%, 18%, 19%, 20%, 21%, 22%, 23%, 24%, or 25%). The plurality of SNPs can comprise or comprise about 5000, 6000, 7000, 8000, 9000, 10000, 11000 (e.g., 11724), 12000, 13000, 14000, 15000, 16000, 17000, 18000, 19000, 20000, 22500, 25000, 27500, 30000, 40000, 50000, or more, SNPs.

In some embodiments, the computing system can determine the BAF value of the SNP for each of the plurality of control samples using absence (and/or presence) of a label (or signal) at (or corresponding to, from, or mapped to) the position of the SNP in the GM data generated from the control sample. For OGM, labels can be fluorescent labels, and signals can be fluorescent signals. For EGM, signals can be electric signals. The presence of a label (which can be a non-fluorescent label) can result in a change in the electric signal. In some embodiments, to determine the BAF value of the SNP for each of the plurality of control samples, the computing system can determine a signal strength of a B-allele of the SNP in the GM data generated from the control sample (or the GM data for the control sample). The computing system can determine the BAF value of the SNP for each of the plurality of control samples using the signal strength of the B-allele of the SNP for the control sample. The computing system can determine the signal strength of the B-allele of the SNP in the GM data generated from the control sample using absence (and/or presence) of a label (or signal) at the position of the SNP in the GM data generated from the control sample.

The signal strength of the B-allele of the SNP for the control sample can be a ratio of (i) a number of deoxyribonucleic acid (DNA) molecules (or fragments) comprising the SNP and without a label (e.g., a fluorescent label for OGM, or a label that is not fluorescent for EGM) at the position of the SNP in the GM data generated from the control sample and (ii) a number of DNA molecules (or fragments) comprising the SNP in the GM data generated from the control sample. The signal strength of the B-allele of the SNP for the control sample can be a percentage of deoxyribonucleic acid (DNA) molecules (or fragments) comprising the SNP in the GM data generated from the control sample and without a label (e.g., a fluorescent label for OGM, or a label that is not fluorescent for EGM) at the position of the SNP. The signal strength of the B-allele of the SNP for the control sample can be 1 minus a ratio of (i) a number of DNA molecules (or fragments) comprising the SNP and with a label (e.g., a fluorescent label for OGM, or a label that is not fluorescent for EGM) at the position of the SNP in the GM data generated from the test sample and (ii) a number of DNA molecules (or fragments) comprising the SNP in the GM data generated from the test sample. The signal strength of the B-allele of the SNP for the control sample can be 1 minus a percentage of DNA molecules (or fragments) comprising the SNP in the GM data generated from the control sample and with a label (e.g., a fluorescent label for OGM, or a label that is not fluorescent for EGM) at the position of the SNP.

A (or each) DNA molecule (or fragment) can be about 150 kilobases (kbp) in length. A (or each) DNA molecule (or fragment) can be at least 150 kilobases (kbp) in length (such as 250 kbp, 500 kbp, 750 kbp, 1 megabases (Mbp), 2 Mbp, or longer, in length). A (or each) DNA molecule (or fragment) can comprise of at least 7, 8, 9, 10, 11, 12, 13, 14, 15, 16, 17, 18, 19, 20, 25, 30, 40, 50, 75, 100, or more) labels. For OGM, labels can be fluorescent labels, and signals can be fluorescent signals. For EGM, signals can be electric signals. The presence of a label (which can be a non-fluorescent label) can result in a change in the electric signal.

In some embodiments, the SNP is at a region with a copy number (CN) loss or a copy number gain. The SNP can be at a region with 0% loss of one copy. The SNP can be at a region with 50% loss of one copy. The SNP can be at a region with complete loss of one copy. The SNP can be at a region with 50% trisomy. The SNP can be at a region with complete trisomy. The SNP can be at a region with complete tetrasomy. To determine the BAF value of the SNP for each of the plurality of control samples, the computing system can determine a signal strength of a B-allele of the SNP in the GM data generated from the control sample (or the GM data for the control sample) using a number of deoxyribonucleic acid (DNA) molecules comprising the SNP mapped to a loss map, a number of DNA molecules comprising the SNP mapped to all maps, and/or a number of DNA molecules mapped to a gain/duplicate map. The computing system can determine the BAF value of the SNP for each of the plurality of control samples using the signal strength of the B-allele of the SNP for the control sample.

The method 600 proceeds to block 612 to block 616, where the computing system clusters the BAF values of the SNP for control samples of the plurality of control samples into a plurality of clusters each comprising (or has or is associated with) a cluster center. A sample (e.g., a control sample or a test sample) can comprise cells, cell-free DNA, cell-free fetal DNA, amniotic fluid, a blood sample, a bone marrow sample, a biopsy sample, or a combination thereof.

The computing system can cluster the BAF values of the SNP for control samples of the plurality of control samples into the plurality of clusters using connectivity-based clustering (e.g., hierarchical clustering), centroid-based clustering (e.g., k-means clustering), distribution-based clustering (e.g., Gaussian mixture model clustering), density-based clustering, grid-based clustering, or a combination thereof. The clustering can be based on a connectivity model (e.g., hierarchical clustering), a centroid model (e.g., k-means clustering), a distribution model (e.g., expectation-maximization), a density model (e.g., DBSCAN and OPTICS), a subspace model (e.g., biclustering), a group model, a graph-based model, a signed graph model, a neural model (e.g., unsupervised neural network), Principal Component Analysis, Independent Component Analysis, or a combination thereof.

The plurality of clusters can comprise 2, 3, 4, 5, 6, 7, 8, 9, 10 or more clusters. A cluster can comprise 0, 1, 2, 3, 4, 5, 6, 7, 8, 9, 10, 20, 30, 40, 50, 75, 100, 250, 500, 750, 1000, 2500, 5000, 10000, 25000, 50000, or more BAF values. A cluster center can have a value of, or of about, 0, 0.05, 0.1, 0.2, 0.3, 0.4, 0.5, 0.6, 0.7, 0.8, 0.9, 0.95, or 1 (on a scale of 0 to 1 where AA, AB, and BB have BAF values of 0, 0.5, and 1 respectively in an idealized situation).

The plurality of clusters can comprise three clusters representing AA, AB, and BB of the SNP. AA, AB, and BB can be genotypes in some embodiments. The three cluster centers of the three clusters representing AA, AB, and BB (which can be, for example, genotypes) can be at about 0, 0.5, and 1.0 respectively. The three cluster centers representing AA, AB, and BB may not be at 0, 0.5, and 1.0 respectively. The computing system can determine a cluster center of each of the plurality of clusters. A cluster center of a cluster of the plurality of cluster can be an average, a mean, a median, or a combination thereof, of the BAF values in the cluster.

In some embodiments, a cluster of the plurality of clusters representing BB (or BB genotype) for a SNP comprises an insufficient number of BAF values (e.g., 20, 19, 18, 17, 16, 15, 14, 13, 12, 11, 10, 9, 8, 7, 6, 5, 4, 3, 2 1, or 0 BAF values). The cluster center of the cluster comprising an insufficient number of BAF values can comprise a measure of cluster centers representing BB (or BB genotypes) for two or more (e.g., 2, 3, 4, 5, 6, 7, 8, 9, 10, or more, or all) of the SNPs (or the plurality of SNPs) with sufficient numbers of BAF values. The measure of cluster centers representing BB (or BB genotypes) can be an average, a mean, a median, or a combination thereof, of the cluster centers representing BB (or BB genotypes).

The method 600 proceeds from block 616 to block 620, where the computing system receives GM data generated from a test sample obtained from a test subject. In some embodiments, the GM data generated from the test sample obtained from the test subject comprises a deoxyribonucleic acid (DNA) consensus map for the test subject. The DNA consensus map can comprise presence and/or absence of labels (or signals) at (or corresponding to, from, or mapped to) the position of the SNP. For OGM, labels can be fluorescent labels, and signals can be fluorescent signals. For EGM, signals can be electric signals. The presence of a label (which can be a non-fluorescent label) can result in a change in the electric signal.

The method 600 proceeds from block 620 to block 624, where the computing system determines a BAF value (an unnormalized or non-normalized BAF value) of the SNP for the test sample using the GM data of the test sample. A BAF value can be or be about 0, 0.05, 0.1, 0.2, 0.3, 0.4, 0.5, 0.6, 0.7, 0.8, 0.9, 0.95, or 1 (on a scale of 0 to 1 where AA, AB, and BB have BAF values of 0, 0.5, and 1 respectively in an idealized situation).

A label for GM (e.g., a fluorescent label for OGM, or a label that is not fluorescent for EGM) can be attached to a predetermined sequence. The gene (or a reference genome sequence) can comprise the predetermined sequence. The SNP can be present at a position (e.g., position 1, 2, 3, 4, 5, 6, 7, 8, 9, or 10) in the predetermined sequence in the gene (or a reference genome sequence). The SNP can overlap the predetermined sequence in the gene (or a reference genome sequence). The nucleobase at the position in the predetermined sequence corresponds to (or is) an A-allele of the SNP. The presence of the label at the SNP in the GM data indicates an A-allele of the SNP. The absence of the label at the SNP in the GM data indicates a B-allele of the SNP. The predetermine sequence can six nucleotides (or 5, 6, 7, 8, 9, 10, or more nucleotides) in length. The predetermine sequence can comprise 5′-CTTAAG-3′. The predetermined sequence can be a recognition sequence of a methyltransferase. The methyltransferase can be a direct labeling enzyme (DLE-1).

The computing system can determine the BAF value of the SNP for the test sample using absence (and/or presence) of a label (or signal) at (or corresponding to, from, or mapped to) the position of the SNP in the GM data generated from the test sample. For OGM, a label can be a fluorescent label, and a signal can be a fluorescent signal. For EGM, a signal can be an electric signal. The presence of a label (which can be a non-fluorescent label) can result in a change in the electric signal. In some embodiments, to determine the BAF value of the SNP for the test sample, the computing system can determine a signal strength of a B-allele of the SNP in the GM data generated from the test sample (or in the GM data for the test sample). To determine the BAF value of the SNP for the test sample, the computing system can determine the BAF value of the SNP for the test sample using the signal strength of the B-allele of the SNP for the test sample. In some embodiments, the computing system can determine the signal strength of the B-allele of the SNP in the GM data generated from the test sample using absence (and/or presence) of a label (or signal) at the position of the SNP in the GM data generated from the test sample.

The signal strength of the B-allele of the SNP for the test sample can be a ratio of (i) a number of deoxyribonucleic acid (DNA) molecules comprising the SNP and without a label (e.g., a fluorescent label for OGM, or a label that is not fluorescent for EGM) at the position of the SNP in the GM data generated from the test sample and (ii) a number of DNA molecules comprising the SNP in the GM data generated from the test sample. The signal strength of the B-allele of the SNP for the test sample can be a percentage of DNA molecules comprising the SNP in the GM data generated from the test sample and without a label (e.g., a fluorescent label for OGM, or a label that is not fluorescent for EGM) at the position of the SNP. The signal strength of the B-allele of the SNP for the test sample can be 1 minus a ratio of (i) a number of DNA molecules comprising the SNP and with a label (e.g., a fluorescent label for OGM, or a label that is not fluorescent for EGM) at the position of the SNP in the GM data generated from the test sample and (ii) a number of DNA molecules comprising the SNP in the GM data generated from the test sample. The signal strength of the B-allele of the SNP for the test sample can be 1 minus a percentage of DNA molecules comprising the SNP in the GM data generated from the test sample and with a label (e.g., a fluorescent label for OGM, or a label that is not fluorescent for EGM) at the position of the SNP.

In some embodiments, to determine the BAF value of the SNP for the test sample, the computing system can determine a signal strength of a B-allele of the SNP in the GM data generated from the test sample (or GM data for the test sample) using a number of deoxyribonucleic acid (DNA) molecules comprising the SNP mapped to a loss map, a number of DNA molecules comprising the SNP mapped to all maps, and/or a number of DNA molecules mapped to a gain/duplicate map. The computing system can determine the BAF value of the SNP for the test sample using the signal strength of the B-allele of the SNP for the test sample.

The method 600 proceeds from block 624 to block 628, where the computing system determines a normalized BAF value of the SNP for the test sample from the BAF value of the SNP for the test sample using one or more (e.g., 2, 3, 4, 5, or more) of the cluster centers (or using values of one or more of the cluster centers). A normalized BAF value can be, or be about, 0, 0.05, 0.1, 0.2, 0.3, 0.4, 0.5, 0.6, 0.7, 0.8, 0.9, 0.95, or 1 (on a scale of 0 to 1 where AA, AB, and BB have BAF values of 0, 0.5, and 1 respectively in an idealized situation).

In some embodiments, the computing system can determine the normalized BAF value of the SNP for the test sample from the BAF value of the SNP for the test sample using two of the cluster centers (or values of two of the cluster centers). In some embodiments, the BAF value of the SNP is smaller than the cluster center of the cluster representing AB (or AB genotype). The normalized BAF value of the SNP can be a ratio of (i) a distance between the cluster center of the cluster representing AA (or AA genotype) and the BAF value of the SNP, and (ii) two times a distance between the cluster center of the cluster representing AA (or AA genotype) and between the cluster center of the cluster representing AB (or AB genotype). In some embodiments, the BAF value of the SNP is greater than the cluster center of the cluster representing AB (or AB genotype). The normalized BAF value of the SNP can be one minus a ratio of (i) a distance between the cluster center of the cluster representing AA (or AA genotype) and the BAF value of the SNP, and (ii) two times a distance between the cluster center of the cluster representing AA (or AA genotype) and between the cluster center of the cluster representing AB (or AB genotype).

In some embodiments, the BAF value of the SNP can be smaller than 0. The normalized BAF value of the SNP can be set to 0. In some embodiments, the BAF value of the SNP can be larger than 1. The normalized BAF value of the SNP can be set to 1.

Cluster Quality. In some embodiments, the computing system can determine a separation between a pair (or each pair) of clusters of the plurality of clusters for a second SNP of the SNPs (or the plurality of SNPs). For example, the computing system can determine a separation between a pair of clusters of the plurality of clusters for a second SNP (e.g., a low-quality SNP) of is below a separation threshold. For example, the computing system can determine a separation between cluster centers of two clusters for a second SNP (e.g., a low-quality SNP) is below a separation threshold. The separation threshold can be, for example, 0.1, 0.15, 0.2, 0.25, 0.3, 0.35, 0.4, or 0.45 on a scale where AA, AB, and BB have BAF values of 0, 0.5, and 1 respectively in an idealized situation. The separation can comprise a Silhouette score, which can be between −1 and 1. The separation threshold can be 0.2, 0.3, 0.4, 0.5, 0.6, 0.7, 0.8, or 0.9. The computing system can remove the second SNP from BAF value and normalized BAF value determination. The computing system can calculate a loss of heterozygosity (LOH) without using the second SNP, without using the BAF value of the second SNP, and/or without using the normalized BAF value of the second SNP. In some embodiments, the computing system can calculate a loss of heterozygosity (LOH) for the test sample using the normalized BAF values of two or more of the SNPs (or the plurality of SNPs).

Split-Label Enhancement. The computing system can perform split-label enhancement. For example, a label (such as a fluorescent label for OGM, or a label that is not fluorescent for EGM) can be assigned to two reference label positions for at least a predetermined percentage (e.g., 5%, 6%, 7%, 8%, 9%, 10%, 11%, 12%, 13%, 14%, 15%, or more) of DNA molecules comprising a third SNP (or split label SNP) the SNPs (or the plurality of SNPs). Such assignment can be present in the GM data generated from a control sample. Such assignment can be present in the GM data generated from two or more of the plurality of control samples. Such assignment can be present in the GM data generated from each of the plurality of control samples. Such assignment can be present in the GM data generated from the test sample. The computing system can remove the third SNP from BAF value and normalized BAF value determination. The computing system can calculate a loss of heterozygosity (LOH) without using the third SNP, the BAF value of the third SNP, and/or the normalized BAF value of the third SNP.

Adjustment. In some embodiments, the computing system can perform per sample adjustment. For example, corresponding clusters of the pluralities of clusters can represent a genotype. The computing system can cluster BAF values of SNPs for the test sample into a plurality of test sample clusters, representing the genotypes, each comprising a test sample cluster center. The computing system can determine a measure of the cluster centers of the clusters representing each of the genotypes. The measure of the cluster centers can be an average, a mean, a median, or a combination thereof, of the cluster centers.

In some embodiments, the computing system can determine a difference between a (or each) test sample cluster center and the measure of the cluster centers of the clusters representing an identical genotype. The computing system can, for a (or each) SNP of the SNPs, adjust a (or each) cluster center of the cluster of the plurality of clusters for the SNP based on the difference to determine an adjusted cluster center. The computing system can determine the normalized BAF value of the SNP for the test sample from the BAF value of the SNP for the test sample using one or more of the plurality of adjusted cluster centers. In some embodiments, the plurality of test sample clusters comprises three test sample clusters representing AA, AB, and BB of the SNP.

Output. In some embodiments, the method comprises: creating a file or a report and/or generating a user interface (UI) comprising a UI element. The UI element can represent or comprise (i) the BAF value of the SNP for one, one or more, or each, of the plurality of control samples, (ii) the signal strength of the B-allele of the SNP for one, one or more, or each, of the plurality of control samples, (iii) the BAF value of the SNP for the test sample, (iv) the signal strength of the B-allele of the SNP for the test sample, and/or (v) the normalized BAF value of the SNP for the test sample. The UI element can comprise a plot representing some or all of the values and signal strengths. A UI element can be a window (e.g., a container window, browser window, text terminal, child window, or message window), a menu (e.g., a menu bar, context menu, or menu extra), an icon, or a tab. A UI element can be for input control (e.g., a checkbox, radio button, dropdown list, list box, button, toggle, text field, or date field). A UI element can be navigational (e.g., a breadcrumb, slider, search field, pagination, slider, tag, icon). A UI element can informational (e.g., a tooltip, icon, progress bar, notification, message box, or modal window). A UI element can be a container (e.g., an accordion).

The method 600 ends at block 632.

### Execution Environment

FIG. 7 depicts a general architecture of an example computing device 700 configured to execute the processes and implement the features described herein. The general architecture of the computing device 700 depicted in FIG. 7 includes an arrangement of computer hardware and software components. The computing device 700 may include many more (or fewer) elements than those shown in FIG. 7. It is not necessary, however, that all of these generally conventional elements be shown in order to provide an enabling disclosure. As illustrated, the computing device 700 includes a processing unit 710, a network interface 720, a computer readable medium drive 730, an input/output device interface 740, a display 750, and an input device 760, all of which may communicate with one another by way of a communication bus. The network interface 720 may provide connectivity to one or more networks or computing systems. The processing unit 710 may thus receive information and instructions from other computing systems or services via a network. The processing unit 710 may also communicate to and from memory 770 and further provide output information for an optional display 750 via the input/output device interface 740. The input/output device interface 740 may also accept input from the optional input device 760, such as a keyboard, mouse, digital pen, microphone, touch screen, gesture recognition system, voice recognition system, gamepad, accelerometer, gyroscope, or other input device.

The memory 770 may contain computer program instructions (grouped as modules or components in some embodiments) that the processing unit 710 executes in order to implement one or more embodiments. The memory 770 generally includes RAM, ROM and/or other persistent, auxiliary or non-transitory computer-readable media. The memory 770 may store an operating system 772 that provides computer program instructions for use by the processing unit 710 in the general administration and operation of the computing device 700. The memory 770 may further include computer program instructions and other information for implementing aspects of the present disclosure.

For example, in one embodiment, the memory 770 includes a BAF value determination 774 for determining a BAF value (e.g., a normalized BAF value), such as the method 600 described with reference to FIG. 6. In addition, memory 770 may include or communicate with the data store 790 and/or one or more other data stores that store input, intermediate results, and results of any method described herein. For example, memory 770 may include or communicate with the data store 790 and/or one or more other data stores that store one or more of: GM data generated from a control sample obtained from a control subject (or each control subject), the BAF value of the SNP for one, one or more, or each, of the plurality of control samples, signal strength of the B-allele of the SNP for one, one or more, or each, of the plurality of control samples, GM data generated from a test sample obtained from a test subject, the BAF value of the SNP for the test sample, the signal strength of the B-allele of the SNP for the test sample, and the normalized BAF value of the SNP for the test sample.

### Additional Considerations

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C can include a first processor configured to carry out recitation A and working in conjunction with a second processor configured to carry out recitations B and C. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example a combination of a DSP and a microprocessor, a plurality of microprocessors, one or more microprocessors in conjunction with a DSP core, or any other such configuration. Although described herein primarily with respect to digital technology, a processor may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure and protected by the following claims.

What is claimed is:

1. A method for determining a normalized B-allele frequency (BAF) value from genome mapping (GM) data comprising:
under control of a hardware processor:
receiving genome mapping (GM) data generated from a plurality of control samples obtained from a plurality of control subjects;
determining a B-allele frequency (BAF) value of a single nucleotide polymorphism (SNP) of a gene for each of the plurality of control samples using the GM data generated from control sample;
clustering the BAF values of the SNP of the gene for control samples of the plurality of control samples into a plurality of clusters each comprising a cluster center;
receiving GM data generated from a test sample obtained from a test subject;
determining a BAF value of the SNP of the gene for the test sample using the GM data of the test sample; and
determining a normalized BAF value of the SNP of the gene for the test sample from the BAF value of the SNP of the gene for the test sample using one or more of the cluster centers.

2. A method for determining normalized B-allele frequency (BAF) values from genome mapping (GM) data comprising:
under control of a hardware processor:
receiving genome mapping (GM) data generated from a plurality of control samples obtained from a plurality of control subjects;
determining a B-allele frequency (BAF) value of each single nucleotide polymorphism (SNP) of SNPs of a plurality of SNPs for each of the plurality of control samples using the GM data generated from control sample;
clustering the BAF values of the SNP for control samples of the plurality of control samples into a plurality of clusters each comprising a cluster center;
receiving GM data generated from a test sample obtained from a test subject;
determining a BAF value of the SNP for the test sample using the GM data of the test sample; and
determining a normalized BAF value of the SNP for the test sample from the BAF value of the SNP for the test sample using one or more of the cluster centers.

3. The method of any one of claims 1-2, wherein receiving the GM data generated from the plurality of control samples obtained from the plurality of control subjects comprises: generating the GM data from a control sample obtained from a control subject.

4. The method of any one of claims 1-3, wherein the GM data generated from a control sample obtained from a control subject comprises a deoxyribonucleic acid (DNA) consensus map for the control subject, optionally wherein the DNA consensus map comprises presence and/or absence of labels at the position of the SNP.

5. The method of any one of claims 1-4, wherein a label for GM is attached to a predetermined sequence, wherein the gene comprises the predetermined sequence, wherein the SNP is present at a position in the predetermined sequence in the gene, optionally wherein the nucleobase at the position in the predetermined sequence corresponds to an A-allele of the SNP, optionally wherein the predetermine sequence is six nucleotides in length, optionally wherein the predetermine sequence comprises 5′-CTTAAG-3′, optionally wherein the predetermined sequence is a recognition sequence of a methyltransferase, optionally wherein the methyltransferase comprises DLE-1.

6. The method of any one of claims 1-5, further comprising: determining the plurality of SNPs, wherein each of the plurality of SNPs overlaps the predetermined sequence.

7. The method of any one of claims 2-6, wherein the plurality of SNPs comprises some or all SNPs in a reference genome sequence of a species of the test subject with a minor allele frequency (MAF) of more than 15%, and/or wherein the plurality of SNPs comprises or comprises about 11724 SNPs.

8. The method of any one of claims 1-7, wherein determining the BAF value of the SNP for each of the plurality of control samples comprises: determining the BAF value of the SNP using absence of a label at the position of the SNP in the GM data generated from the control sample.

9. The method of any one of claims 1-8, wherein determining the BAF value of the SNP for each of the plurality of control samples comprises:
determining a signal strength of a B-allele of the SNP in the GM data generated from the control sample; and
determining the BAF value of the SNP for the control sample using the signal strength of the B-allele of the SNP for the control sample.

10. The method of claim 9,
wherein determining the signal strength of the B-allele of the SNP in the GM data generated from the control sample comprises: determining the signal strength of the B-allele of the SNP in the GM data generated from the control sample using absence of a label at the position of the SNP in the GM data generated from the control sample, and/or
wherein the signal strength of the B-allele of the SNP for the control sample is a ratio of (i) a number of deoxyribonucleic acid (DNA) molecules comprising the SNP and without a label at the position of the SNP in the GM data generated from the control sample and (ii) a number of DNA molecules comprising the SNP in the GM data generated from the control sample.

11. The method of any one of claims 2-10, further comprising:
determining a separation between a pair of clusters of the plurality of clusters for a second SNP of the SNPs is below a separation threshold, optionally wherein the separation comprises a Silhouette score; and
removing the second SNP from BAF value and normalized BAF value determination, and/or calculating a loss of heterozygosity (LOH) without using the second SNP, the BAF value of the second SNP, and/or the normalized BAF value of the second SNP.

12. The method of any one of claims 2-11, further comprising: calculating a loss of heterozygosity (LOH) for the test sample using the normalized BAF values of two or more of the SNPs.

13. The method of any one of claims 2-12, wherein a label is assigned to two reference label positions for at least a predetermined percentage of DNA molecules comprising a third SNP the SNPs, the method further comprising: removing the third SNP from BAF value and normalized BAF value determination, and/or calculating a loss of heterozygosity (LOH) without using the third SNP, the BAF value of the third SNP, and/or the normalized BAF value of the third SNP.

14. The method of any one of claims 1-13, wherein the SNP is at a region with a copy number (CN) loss or a copy number gain, optionally wherein the SNP is at a region with 0% loss of one copy, 50% loss of one copy, complete loss of one copy, 50% trisomy, complete trisomy, or complete tetrasomy, and wherein determining the BAF value of the SNP for each of the plurality of control samples comprises:
determining a signal strength of a B-allele of the SNP in the GM data generated from the control sample using a number of deoxyribonucleic acid (DNA) molecules comprising the SNP mapped to a loss map, a number of DNA molecules comprising the SNP mapped to all maps, and/or a number of DNA molecules mapped to a gain/duplicate map; and
determining the BAF value of the SNP for the control sample using the signal strength of the B-allele of the SNP for the control sample.

15. The method of any one of claims 1-14, wherein clustering the BAF values comprises: clustering the BAF values of the SNP for control samples of the plurality of control samples into the plurality of clusters using connectivity-based clustering, centroid-based clustering, distribution-based clustering, density-based clustering, grid-based clustering, or a combination thereof.

16. The method of any one of claims 1-15, wherein the plurality of clusters comprises three clusters representing AA, AB, and BB genotypes of the SNP, optionally wherein the three cluster centers of the three clusters representing AA, AB, and BB genotypes are at about 0, 0.5, and 1.0 respectively, optionally wherein the three cluster centers representing AA, AB, and BB are not at 0, 0.5, and 1.0 respectively.

17. The method of any one of claims 1-16, further comprising: determining a cluster center of each of the plurality of clusters.

18. The method of any one of claims 1-17, wherein a cluster center of a cluster of the plurality of cluster is an average, a mean, a median, or a combination thereof, of the BAF values in the cluster.

19. The method of any one of claims 1-18, wherein a cluster of the plurality of clusters representing BB genotype for a SNP comprises an insufficient number of BAF values, and wherein the cluster center of the cluster comprising an insufficient number of BAF values comprises a measure of cluster centers representing BB genotypes for two or more of the SNPs with sufficient numbers of BAF values, optionally wherein the measure of cluster centers representing BB genotypes is an average, a mean, a median, or a combination thereof, of the cluster centers representing BB genotypes.

20. The method of any one of claims 1-19, wherein receiving the GM data generated from the test sample obtained from the test subject comprises: generating the GM data from the test sample obtained from the test subject.

21. The method of any one of claims 1-20, wherein the GM data generated from the test sample obtained from the test subject comprises a deoxyribonucleic acid (DNA) consensus map for the test subject, optionally wherein the DNA consensus map comprises presence and/or absence of labels at the position of the SNP.

22. The method of any one of claims 1-21, wherein determining the BAF value of the SNP for the test sample comprises: determining the BAF value of the SNP using absence of a label at the position of the SNP in the GM data generated from the test sample.

23. The method of any one of claims 1-22, wherein determining the BAF value of the SNP for the test sample comprises:
determining a signal strength of a B-allele of the SNP in the GM data generated from the test sample; and
determining the BAF value of the SNP for the test sample using the signal strength of the B-allele of the SNP for the test sample.

24. The method of claim 23,
wherein determining the signal strength of the B-allele of the SNP in the GM data generated from the test sample comprises: determining the signal strength of the B-allele of the SNP in the GM data generated from the test sample using absence of a label at the position of the SNP in the GM data generated from the test sample, and/or
wherein the signal strength of the B-allele of the SNP for the test sample is a ratio of (i) a number of deoxyribonucleic acid (DNA) molecules comprising the SNP and without a label at the position of the SNP in the GM data generated from the test sample and (ii) a number of DNA molecules comprising the SNP in the GM data generated from the test sample.

25. The method of any one of claims 1-24, wherein determining the BAF value of the SNP for the test sample comprises:
determining a signal strength of a B-allele of the SNP in the GM data generated from the test sample using a number of deoxyribonucleic acid (DNA) molecules comprising the SNP mapped to a loss map, a number of DNA molecules comprising the SNP mapped to all maps, and/or a number of DNA molecules mapped to a gain/duplicate map; and
determining the BAF value of the SNP for the test sample using the signal strength of the B-allele of the SNP for the test sample.

26. The method of any one of claims 1-25, wherein determining the normalized BAF value for the test sample comprises: determining the normalized BAF value of the SNP for the test sample from the BAF value of the SNP for the test sample using two cluster centers of the plurality of cluster centers, or cluster centers of two cluster centers of the plurality of cluster centers.

27. The method claim 26, wherein the BAF value of the SNP is smaller than the cluster center of the cluster representing AB genotype, and wherein the normalized BAF value of the SNP is a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

28. The method claim 26, wherein the BAF value of the SNP is greater than the cluster center of the cluster representing AB genotype, and wherein the normalized BAF value of the SNP is one minus a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

29. The method of any one of claims 2-28, wherein corresponding clusters of the pluralities of clusters represent a genotype, the method further comprising:
clustering BAF values of SNPs for the test sample into a plurality of test sample clusters, representing the genotypes, each comprising a test sample cluster center;
determining a measure of the cluster centers of the clusters representing each of the genotypes, optionally wherein the measure of the cluster centers is an average, a mean, a median, or a combination thereof, of the cluster centers;
determining a difference between a test sample cluster center and the measure of the cluster centers of the clusters representing an identical genotype; and
for a SNP of the SNPs, adjusting a cluster center of the cluster of the plurality of clusters for the SNP based on the difference to determine an adjusted cluster center,
wherein determining the normalized BAF value of the SNP for the test sample comprises: determining the normalized BAF value of the SNP for the test sample from the BAF value of the SNP for the test sample using one or more of the plurality of adjusted cluster centers.

30. The method of claim 29, wherein the plurality of test sample clusters comprises three test sample clusters representing AA, AB, and BB genotypes of the SNP.

31. The method of any one of claims 1-30, comprising: creating a file or a report and/or generating a user interface (UI) comprising a UI element representing or comprising (i) the BAF value of the SNP for one, one or more, or each, of the plurality of control samples, (ii) the signal strength of the B-allele of the SNP for one, one or more, or each, of the plurality of control samples, (iii) the BAF value of the SNP for the test sample, (iv) the signal strength of the B-allele of the SNP for the test sample, and/or (v) the normalized BAF value of the SNP for the test sample.

32. The method of any one of claims 1-31, wherein the sample comprises cells, cell-free DNA, cell-free fetal DNA, amniotic fluid, a blood sample, a bone marrow sample, a biopsy sample, or a combination thereof.

33. The method of any one of claims 1-32, wherein the GM data comprises optical genome mapping (OGM) data.

34. The method of any one of claims 4-33, wherein the label comprises a fluorescent label, and/or wherein the labels comprise fluorescent labels.

35. The method of any one of claims 1-32, wherein the GM data comprises electronic genome mapping (EGM) data.

36. The method of any one of claims 4-32 and 35, wherein the label comprises a label that is not fluorescent, and/or wherein the labels comprise labels that are not fluorescent.

37. A system for determining a normalized B-allele frequency (BAF) value from genome mapping (GM) data comprising:
non-transitory memory configured to store:
executable instructions,
genome mapping (GM) data generated from a plurality of control samples obtained from a plurality of control subjects,
a B-allele frequency (BAF) value of a single nucleotide polymorphism (SNP) of a gene for each of the plurality of control samples determined using the GM data generated from control sample, and/or
a plurality of clusters, each comprising a cluster center, generated from BAF values of the SNP of the gene for control samples of the plurality of control samples; and
a hardware processor in communication with the non-transitory memory, the hardware processor programmed by the executable instructions to perform:
receiving GM data generated from a test sample obtained from a test subject;
determining a BAF value of the SNP of the gene for the test sample using the GM data of the test sample; and
determining a normalized BAF value of the SNP of the gene for the test sample from the BAF value of the SNP of the gene for the test sample using one or more of the cluster centers.

38. The system of claim 37, wherein the hardware processor is programmed by the executable instructions to perform:
receiving the GM data generated from the plurality of control samples obtained from the plurality of control subjects;
determining the BAF value of the SNP of the gene for each of the plurality of control samples using the GM data generated from control sample; and
clustering BAF values of the SNP of the gene for control samples of the plurality of control samples into a plurality of clusters, each comprising a cluster center.

39. A system for determining a normalized B-allele frequency (BAF) values from genome mapping (GM) data comprising:
non-transitory memory configured to store:
executable instructions,
genome mapping (GM) data generated from a plurality of control samples obtained from a plurality of control subjects,
a B-allele frequency (BAF) value of each single nucleotide polymorphism (SNP) of SNPs of a plurality of SNPs for each of the plurality of control samples determined using the GM data generated from control sample, and/or
a plurality of clusters, each comprising a cluster center, generated from BAF values of the SNP for control samples of the plurality of control samples; and
a hardware processor in communication with the non-transitory memory, the hardware processor programmed by the executable instructions to perform:
receiving GM data generated from a test sample obtained from a test subject;
determining a BAF value of the SNP for the test sample using the GM data of the test sample; and
determining a normalized BAF value of the SNP for the test sample from the BAF value of the SNP for the test sample using one or more of the cluster centers.

40. The system of claim 39, wherein the hardware processor is programmed by the executable instructions to perform:
receiving the GM data generated from the plurality of control samples obtained from the plurality of control subjects;
determining the BAF value of each SNP of the SNPs of the plurality of SNPs for each of the plurality of control samples using the GM data generated from control sample; and
clustering BAF values of the SNP for control samples of the plurality of control samples into a plurality of clusters each comprising a cluster center.

41. The system of any one of claims 38-40, wherein receiving the GM data generated from the plurality of control samples obtained from the plurality of control subjects comprises: generating the GM data from a control sample obtained from a control subject.

42. The system of any one of claims 37-41, wherein the GM data generated from a control sample obtained from a control subject comprises a deoxyribonucleic acid (DNA) consensus map for the control subject, optionally wherein the DNA consensus map comprises presence and/or absence of labels at the position of the SNP.

43. The system of any one of claims 37-42, wherein a label for GM is attached to a predetermined sequence, wherein the gene comprises the predetermined sequence, wherein the SNP is present at a position in the predetermined sequence in the gene, optionally wherein the nucleobase at the position in the predetermined sequence corresponds to an A-allele of the SNP, optionally wherein the predetermine sequence is six nucleotides in length, optionally wherein the predetermine sequence comprises 5′-CTTAAG-3′, optionally wherein the predetermined sequence is a recognition sequence of a methyltransferase, optionally wherein the methyltransferase comprises DLE-1.

44. The system of any one of claims 37-43, wherein the hardware processor is programmed by the executable instructions to perform: determining the plurality of SNPs, and wherein each of the plurality of SNPs overlaps the predetermined sequence.

45. The system of any one of claims 39-44, wherein the plurality of SNPs comprises some or all SNPs in a reference genome sequence of a species of the test subject with a minor allele frequency (MAF) of more than 15%, and/or wherein the plurality of SNPs comprises comprises or comprises about 11724 SNPs.

46. The system of any one of claims 37-45, wherein the BAF value of the SNP is determined using absence of a label at the position of the SNP in the GM data generated from the control sample.

47. The system of any one of claims 37-46, wherein the BAF value of the SNP for each of the plurality of control samples is determined by:
determining a signal strength of a B-allele of the SNP in the GM data generated from the control sample; and
determining the BAF value of the SNP for the control sample using the signal strength of the B-allele of the SNP for the control sample.

48. The system of claim 47,
wherein determining the signal strength of the B-allele of the SNP in the GM data generated from the control sample comprises: determining the signal strength of the B-allele of the SNP in the GM data generated from the control sample using absence of a label at the position of the SNP in the GM data generated from the control sample, and/or
wherein the signal strength of the B-allele of the SNP for the control sample is a ratio of (i) a number of deoxyribonucleic acid (DNA) molecules comprising the SNP and without a label at the position of the SNP in the GM data generated from the control sample and (ii) a number of DNA molecules comprising the SNP in the GM data generated from the control sample.

49. The system of any one of claims 40-48, wherein the hardware processor is programmed by the executable instructions to perform:
determining a separation between a pair of clusters of the plurality of clusters for a second SNP of the SNPs is below a separation threshold, optionally wherein the separation comprises a Silhouette score; and
removing the second SNP from BAF value and normalized BAF value determination, and/or calculating a loss of heterozygosity (LOH) without using the second SNP, the BAF value of the second SNP, and/or the normalized BAF value of the second SNP.

50. The system of any one of claims 40-49, wherein the hardware processor is programmed by the executable instructions to perform: calculating a loss of heterozygosity (LOH) for the test sample using the normalized BAF values of two or more of the SNPs.

51. The system of any one of claims 40-50, wherein a label is assigned to two reference label positions for at least a predetermined percentage of DNA molecules comprising a third SNP the SNPs, wherein the hardware processor is programmed by the executable instructions to perform: removing the third SNP from BAF value and normalized BAF value determination, and/or calculating a loss of heterozygosity (LOH) without using the third SNP, the BAF value of the third SNP, and/or the normalized BAF value of the third SNP.

52. The system of any one of claims 37-51, wherein the SNP is at a region with a copy number (CN) loss or a copy number gain, optionally wherein the SNP is at a region with 0% loss of one copy, 50% loss of one copy, complete loss of one copy, 50% trisomy, complete trisomy, or complete tetrasomy, and wherein determining the BAF value of the SNP for each of the plurality of control samples comprises:
determining a signal strength of a B-allele of the SNP in the GM data generated from the control sample using a number of deoxyribonucleic acid (DNA) molecules comprising the SNP mapped to a loss map, a number of DNA molecules comprising the SNP mapped to all maps, and/or a number of DNA molecules mapped to a gain/duplicate map; and
determining the BAF value of the SNP for the control sample using the signal strength of the B-allele of the SNP for the control sample.

53. The system of any one of claims 37-52, wherein the plurality of clusters is generated from the BAF values of the SNP for control samples of the plurality of control samples using connectivity-based clustering, centroid-based clustering, distribution-based clustering, density-based clustering, grid-based clustering, or a combination thereof.

54. The system of any one of claims 37-53, wherein the plurality of clusters comprises three clusters representing AA, AB, and BB genotypes of the SNP, optionally wherein the three cluster centers of the three clusters representing AA, AB, and BB genotypes are at about 0, 0.5, and 1.0 respectively, optionally wherein the three cluster centers representing AA, AB, and BB are not at 0, 0.5, and 1.0 respectively.

55. The system of any one of claims 37-54, wherein the hardware processor is programmed by the executable instructions to perform: determining a cluster center of each of the plurality of clusters.

56. The system of any one of claims 37-55, wherein a cluster center of a cluster of the plurality of cluster is an average, a mean, a median, or a combination thereof, of the BAF values in the cluster.

57. The system of any one of claims 37-56, wherein a cluster of the plurality of clusters representing BB genotype for a SNP comprises an insufficient number of BAF values, wherein the cluster center of the cluster comprising an insufficient number of BAF values comprises a measure of cluster centers representing BB genotypes for two or more of the SNPs with sufficient numbers of BAF values, optionally wherein the measure of cluster centers representing BB genotypes is an average, a mean, a median, or a combination thereof, of the cluster centers representing BB genotypes.

58. The system of any one of claims 37-57, wherein receiving the GM data generated from the test sample obtained from the test subject comprises: generating the GM data from the test sample obtained from the test subject.

59. The system of any one of claims 37-58, wherein the GM data generated from the test sample obtained from the test subject comprises a deoxyribonucleic acid (DNA) consensus map for the test subject, optionally wherein the DNA consensus map comprises presence and/or absence of labels at the position of the SNP.

60. The system of any one of claims 37-59, wherein determining the BAF value of the SNP for the test sample comprises: determining the BAF value of the SNP using absence of a label at the position of the SNP in the GM data generated from the test sample.

61. The system of any one of claims 37-60, wherein determining the BAF value of the SNP for the test sample comprises:
determining a signal strength of a B-allele of the SNP in the GM data generated from the test sample; and
determining the BAF value of the SNP for the test sample using the signal strength of the B-allele of the SNP for the test sample.

62. The system of claim 61,
wherein determining the signal strength of the B-allele of the SNP in the GM data generated from the test sample comprises: determining the signal strength of the B-allele of the SNP in the GM data generated from the test sample using absence of a label at the position of the SNP in the GM data generated from the test sample, and/or
wherein the signal strength of the B-allele of the SNP for the test sample is a ratio of (i) a number of deoxyribonucleic acid (DNA) molecules comprising the SNP and without a label at the position of the SNP in the GM data generated from the test sample and (ii) a number of DNA molecules comprising the SNP in the GM data generated from the test sample.

63. The system of any one of claims 37-62, wherein determining the normalized BAF value for the test sample comprises: determining the normalized BAF value of the SNP for the test sample from the BAF value of the SNP for the test sample using two cluster centers of the plurality of cluster centers, or cluster centers of two cluster centers of the plurality of cluster centers.

64. The system claim 63, wherein the BAF value of the SNP is smaller than the cluster center of the cluster representing AB genotype, and wherein the normalized BAF value of the SNP is a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

65. The system claim 63, wherein the BAF value of the SNP is greater than the cluster center of the cluster representing AB genotype, and wherein the normalized BAF value of the SNP is one minus a ratio of (i) a distance between the cluster center of the cluster representing AA genotype and the BAF value of the SNP, and (ii) two times a distance between the cluster center of the cluster representing AA genotype and between the cluster center of the cluster representing AB genotype.

66. The system of any one of claims 39-65, wherein corresponding clusters of the pluralities of clusters represent a genotype, and wherein the hardware processor is programmed by the executable instructions to perform:
clustering BAF values of SNPs for the test sample into a plurality of test sample clusters, representing the genotypes, each comprising a test sample cluster center;
determining a measure of the cluster centers of the clusters representing each of the genotypes, optionally wherein the measure of the cluster centers is an average, a mean, a median, or a combination thereof, of the cluster centers;
determining a difference between a test sample cluster center and the measure of the cluster centers of the clusters representing an identical genotype; and
for a SNP of the SNPs, adjusting a cluster center of the cluster of the plurality of clusters for the SNP based on the difference to determine an adjusted cluster center,
wherein determining the normalized BAF value of the SNP for the test sample comprises: determining the normalized BAF value of the SNP for the test sample from the BAF value of the SNP for the test sample using one or more of the plurality of adjusted cluster centers.

67. The system of claim 66, wherein the plurality of test sample clusters comprises three test sample clusters representing AA, AB, and BB genotypes of the SNP.

68. The system of any one of claims 37-67, wherein the hardware processor is programmed by the executable instructions to perform: creating a file or a report and/or generating a user interface (UI) comprising a UI element representing or comprising (i) the BAF value of the SNP for one, one or more, or each, of the plurality of control samples, (ii) the signal strength of the B-allele of the SNP for one, one or more, or each, of the plurality of control samples, (iii) the BAF value of the SNP for the test sample, (iv) the signal strength of the B-allele of the SNP for the test sample, and/or (v) the normalized BAF value of the SNP for the test sample.

69. The system of any one of claims 37-68, wherein the sample comprises cells, cell-free DNA, cell-free fetal DNA, amniotic fluid, a blood sample, a bone marrow sample, a biopsy sample, or a combination thereof.

70. The system of any one of claims 37-69, wherein the GM data comprises optical genome mapping (OGM) data.

71. The system of any one of claims 42-70, wherein the label comprises a fluorescent label, and/or wherein the labels comprise fluorescent labels.

72. The system of any one of claims 37-69, wherein the GM data comprises electronic genome mapping (EGM) data.

73. The system of any one of claims 42-69 and 72, wherein the label comprises a label that is not fluorescent, and/or wherein the labels comprise labels that are not fluorescent.
